(19) United States
(12) Patent Application Publication
(10) Pub. No.: US 2023/0314418 A1
(43) Pub. Date: Oct. 5, 2023

(54) SINGLE DOMAIN ANTIBODY/POLYMERIC TANDEM FLUORESCENT DYE CONJUGATES, AND METHODS FOR MAKING AND USING THE SAME

(71) Applicant: Becton, Dickinson and Company, Franklin Lakes, NJ (US)

(72) Inventors: David Moureau, San Diego, CA (US); Stephanie Rigaud, San Diego, CA (US)

(21) Appl. No.: 18/126,819

(22) Filed: Mar. 27, 2023

Related U.S. Application Data
(60) Provisional application No. 63/326,104, filed on Mar. 31, 2022.

(51) Int. Cl. G01N 33/533; G01N 33/569; C07K 19/00

(52) U.S. Cl. CPC G01N 33/533; G01N 33/56966; C07K 19/00

(57) ABSTRACT

Single domain antibody/polymeric fluorescent tandem dye conjugates are provided. Aspects of the provided conjugates include: a single domain antibody having one or more polymeric tandem fluorescent dyes conjugated thereto. In some instances, the polymeric tandem fluorescent dye includes: (i) a polymeric backbone made up of non-conjugated repeat units; (ii) a plurality of pendant donor chromophore groups each independently linked to a non-conjugated repeat unit of the polymeric backbone; and (iii) one or more pendant acceptor fluorophores linked to a non-conjugated repeat unit of the polymeric backbone, wherein pendant donor and acceptor fluorophores are in energy transfer relationship. Also provided are methods of using the conjugates, e.g., in methods of evaluating a sample for the presence of a target analyte, and kits comprising the conjugates that find use in embodiments of the methods.

### CROSS-REFERENCE TO RELATED APPLICATION

Pursuant to 35 U.S.C. § 119 (e), this application claims priority to the filing date of U.S. Provisional Patent Application Ser. No. 63/326,104 filed Mar. 31, 2022; the disclosure of which application is incorporated herein by reference in its entirety.

### INTRODUCTION

Fluorescent dyes are compounds which, when irradiated with light of a wavelength which they absorb, emit light of a (usually) different wavelength. Fluorescent dyes find use in a variety of applications in biochemistry, biology and medicine, e.g. in diagnostic kits, in microscopy or in drug screening. Fluorescent dyes are characterized by a number of parameters allowing a user to select a suitable dye depending on the desired purpose. Parameters of interest include the excitation wavelength maximum, the emission wavelength maximum, the Stokes shift, the extinction coefficient, the fluorescence quantum yield and the fluorescence lifetime. Dyes may be selected according to the application of interest in order to, e.g., allow penetration of exciting radiation into biological samples, to minimize background fluorescence and/or to achieve a high signal-to-noise ratio.

Molecular recognition involves the specific binding of two molecules. Molecules which have binding specificity for a target biomolecule find use in a variety of research and diagnostic applications, such as the labelling and separation of analytes, flow cytometry, in situ hybridization, enzyme-linked immunosorbent assays (ELISAs), western blot analysis, magnetic cell separations and chromatography. Target biomolecules may be detected by labelling with a fluorescent dye.

### SUMMARY

Single domain antibody/polymeric fluorescent tandem dye conjugates are provided. Aspects of the provided conjugates include: a single domain antibody having one or more polymeric tandem fluorescent dyes conjugated thereto. In some instances, the polymeric tandem fluorescent dye includes: (i) a polymeric backbone made up of non-conjugated repeat units; (ii) a plurality of pendant donor chromophore groups each independently linked to a non-conjugated repeat unit of the polymeric backbone; and (iii) one or more pendant acceptor fluorophores linked to a non-conjugated repeat unit of the polymeric backbone, wherein pendant donor and acceptor fluorophores are in energy transfer relationship. Also provided are methods of using the conjugates, e.g., in methods of evaluating a sample for the presence of a target analyte, and kits comprising the conjugates that find use in embodiments of the methods.

### BRIEF DESCRIPTION OF THE DRAWINGS

The invention may be best understood from the following detailed description when read in conjunction with the accompanying drawings. Included in the drawings are the following figures:

FIGS. 1A and 1B provide illustrations of a single domain antibody/polymeric fluorescent tandem dye conjugate according to an embodiment of the invention.

FIGS. 2A and 2B provide a schematic of an assay employing a single domain antibody/polymeric fluorescent tandem dye conjugate and a prior art single domain antibody reagent, as described further in the experimental section below.

### DEFINITIONS

As used herein, the terms “chemoselective functional group” and “chemoselective tag” are used interchangeably and refer to a functional group that can selectively react with another compatible functional group to form a covalent bond, in some cases, after optional activation of one of the functional groups. Chemoselective functional groups of interest include, but are not limited to, thiols and maleimide or iodoacetamide, amines and carboxylic acids or active esters thereof, as well as groups that can react with one another via Click chemistry, e.g., azide and alkyne groups (e.g., cyclooctyne groups), tetrazine, transcyclooctene, dienes and dienophiles, and azide, sulfur(VI) fluoride exchange chemistry (SuFEX), sulfonyl fluoride, as well as hydroxyl, hydrazido, hydrazino, aldehyde, ketone, azido, alkyne, phosphine, epoxide, and the like.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

The terms “support bound” and “linked to a support” are used interchangeably and refer to a moiety (e.g., a specific binding member) that is linked covalently or non-covalently to a support of interest. Covalent linking may involve the chemical reaction of two compatible functional groups (e.g., two chemoselective functional groups, an electrophile and a nucleophile, etc.) to form a covalent bond between the two moieties of interest (e.g., a support and a specific binding member). In some cases, non-covalent linking may involve specific binding between two moieties of interest (e.g., two affinity moieties such as a hapten and an antibody or a biotin moiety and a streptavidin, etc.). In certain cases, non-covalent linking may involve absorption to a substrate.

The term “polypeptide” refers to a polymeric form of amino acids of any length, including peptides that range from 2-50 amino acids in length and polypeptides that are greater than 50 amino acids in length. The terms “polypeptide” and “protein” are used interchangeably herein. The term “polypeptide” includes polymers of coded and non-coded amino acids, chemically or biochemically modified or derivatized amino acids, and polypeptides having modified peptide backbones in which the conventional backbone has been replaced with non-naturally occurring or synthetic backbones. A polypeptide may be of any convenient length, e.g., 2 or more amino acids, such as 4 or more amino acids, 10 or more amino acids, 20 or more amino acids, 50 or more amino acids, 100 or more amino acids, 300 or more amino acids, such as up to 500 or 1000 or more amino acids. “Peptides” may be 2 or more amino acids, such as 4 or more amino acids, 10 or more amino acids, 20 or more amino acids, such as up to 50 amino acids. In some embodiments, peptides are between 5 and 30 amino acids in length.

As used herein the term “isolated,” refers to an moiety of interest that is at least 60% free, at least 75% free, at least 90% free, at least 95% free, at least 98% free, and even at least 99% free from other components with which the moiety is associated with prior to purification.

A “plurality” contains at least 2 members. In certain cases, a plurality may have 5 or more, such as 6 or more, 7 or more, 8 or more, 9 or more, 10 or more, or more, 30 or more, 40 or more, 50 or more, 60 or more, 70 or more, 80 or more, 90 or more, 100 or more, 300 or more, 1000 or more, 3000 or more, 10,000 or more, 100,000 or more members.

Numeric ranges are inclusive of the numbers defining the range.

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining,” “measuring,” and “assessing,” and “assaying” are used interchangeably and include both quantitative and qualitative determinations.

The term “separating”, as used herein, refers to physical separation of two elements (e.g., by size or affinity, etc.) as well as degradation of one element, leaving the other intact.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

As used herein, the terms “water solubilizing group”, “water soluble group” and WSG are used interchangeably and refer to a group or substituent that is well solvated in aqueous environments e.g., under physiological conditions, and which imparts improved water solubility upon the molecule to which it is attached. A WSG can increase the solubility of a multichromophore in a predominantly aqueous solution, as compared to a control multichromophore which lacks the WSG. The water solubilizing groups may be any convenient hydrophilic group that is well solvated in aqueous environments.

The terms “polyethylene oxide”, “PEO”, “polyethylene glycol” and “PEG” are used interchangeably and refer to a polymeric group including a chain described by the formula —(CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use in the subject multichromophores include those PEGs described by S. Zalipsky in “Functionalized poly(ethylene glycol) for preparation of biologically relevant conjugates”, Bioconjugate Chemistry 1995, 6 (2), 150-165; and by Zhu et al in “Water-Soluble Conjugated Polymers for Imaging, Diagnosis, and Therapy”, Chem. Rev., 2012, 112 (8), pp 4687-4735.

The term “alkyl” by itself or as part of another substituent refers to a saturated branched or straight-chain monovalent hydrocarbon radical derived by the removal of one hydrogen atom from a single carbon atom of a parent alkane. Alkyl groups of interest include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group includes from 1 to 20 carbon atoms. In some embodiments, an alkyl group includes from 1 to 10 carbon atoms. In certain embodiments, a lower alkyl group includes from 1 to 6 carbon atoms, such as from 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

“Alkenyl” refers to a monoradical, branched or linear, cyclic or non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Alkynyl” or “alkyne” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” or “substituted alkyne” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Heterocyclyl” refers to a monoradical, cyclic group that contains a heteroatom (e.g. O, S, N) in as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Amino” refers to the group —NH₂. The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aryl” by itself or as part of another substituent refers to a monovalent aromatic hydrocarbon radical derived by the removal of one hydrogen atom from a single carbon atom of an aromatic ring system. Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

“Substituted aryl”, unless otherwise constrained by the definition for the aryl substituent, refers to an aryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl, —SO₂-heteroaryl and trihalomethyl.

“Heteroaryl” by itself or as part of another substituent, refers to a monovalent heteroaromatic radical derived by the removal of one hydrogen atom from a single atom of a heteroaromatic ring system. Heteroaryl groups of interest include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, triazole, benzotriazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Substituted heteroaryl”, unless otherwise constrained by the definition for the substituent, refers to an heteroaryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

The term “alkaryl” or “aralkyl” refers to the groups -alkylene-aryl and substituted alkylene-aryl where alkylene, substituted alkylene and aryl are defined herein.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like. “Substituted alkylene” refers to an alkylene group having from 1 to 3 hydrogens replaced with substituents as described for carbons in the definition of “substituted” below.

“Substituted” refers to a group in which one or more hydrogen atoms are independently replaced with the same or different substituent(s). Substituents of interest include, but are not limited to, alkylenedioxy (such as methylenedioxy), -M, —R⁶⁰, —O—, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O—, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group. When the group being substituted is an aryl or heteroaryl group, the substituent(s) (e.g., as described herein) may be referred to as “aryl substituent(s)”.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

“Acyl” refers to a group of formula —C(O)R wherein R is alkyl, alkenyl, or alkynyl. For example, the acetyl group has formula —C(O)CH₃.

“Halo” and “halogen” refer to the chloro, bromo, fluoro, and iodo groups.

“Carboxyl”, “carboxy”, and “carboxylate” refer to the —CO₂H group and salts thereof.

“Sulfonyl” refers to the group —SO₂R, wherein R is alkyl, alkenyl, alkynyl, aryl, heteroaryl, heterocyclyl, and substituted versions thereof. Exemplary sulfonyl groups includes —SO₂CH₃ and —SO₂(C₆H₅).

Unless otherwise specified, reference to an atom is meant to include all isotopes of that atom. For example, reference to H is meant to include ¹H, ²H (i.e., D) and ³H (i.e., T), and reference to C is meant to include ¹²C and all isotopes of carbon (such as ¹³C). In addition, any groups described include all stereoisomers of that group.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

### DETAILED DESCRIPTION

Single domain antibody/polymeric fluorescent tandem dye conjugates are provided. Aspects of the provided conjugates include: a single domain antibody having one or more polymeric tandem fluorescent dyes conjugated thereto. In some instances, the polymeric tandem fluorescent dye includes: (i) a polymeric backbone made up of non-conjugated repeat units; (ii) a plurality of pendant donor chromophore groups each independently linked to a non-conjugated repeat unit of the polymeric backbone; and (iii) one or more pendant acceptor fluorophores linked to a non-conjugated repeat unit of the polymeric backbone, wherein pendant donor and acceptor fluorophores are in energy transfer relationship. Also provided are methods of using the conjugates, e.g., in methods of evaluating a sample for the presence of a target analyte, and kits comprising the conjugates that find use in embodiments of the methods.

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Still, certain terms are defined below for the sake of clarity and ease of reference. Further, although any methods and materials similar or equivalent to those described herein can also be used in the practice or testing of the present invention, representative illustrative methods and materials are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various embodiments of the invention, single domain antibody/polymeric fluorescent tandem dye conjugates are reviewed first in greater detail, followed by a review of methods of using and making the conjugates, as well as a review of kits that include the conjugates.

### Single Domain Antibody/Polymeric Fluorescent Tandem Dye Conjugates

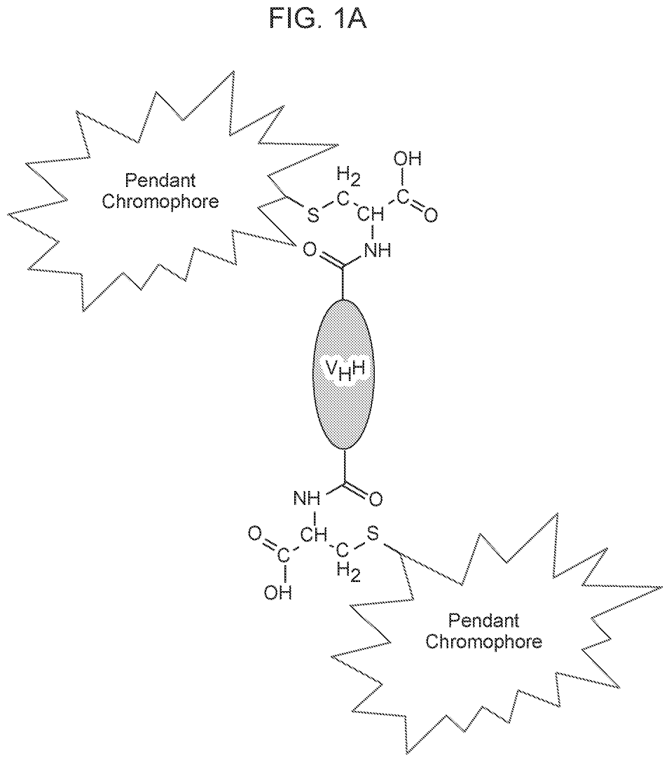

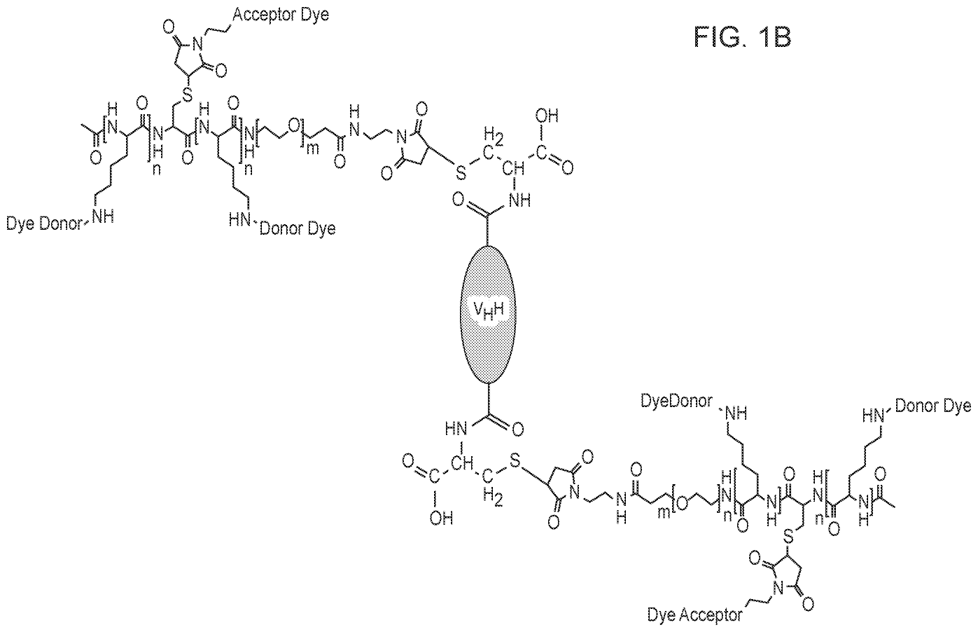

As summarized above, the present disclosure provides single domain antibody/polymeric fluorescent tandem dye conjugates. Dye conjugates of embodiments of the invention include a single domain antibody and one or more polymeric tandem fluorescent dyes conjugated thereto. In other words, the dye conjugates of embodiments of the invention include at least one polymeric tandem fluorescent dye conjugated to a single domain antibody, wherein the number of polymeric tandem fluorescent dyes that are conjugated to a single domain antibody in a given single domain antibody/polymeric fluorescent dye conjugate of the invention may vary, ranging in some instances from 2 to 5, such as 2 to 4, including 2 to 3 polymeric tandem fluorescent dyes. In some instances, single domain antibody/polymeric fluorescent tandem dye conjugates of the invention have a low molecular weight. In some embodiments, the molecular weight of the single domain antibody/polymeric fluorescent tandem dye conjugates ranges from 20 to 200 kDa, such as 25 to 150 kDa, including 30 to 75 kDa, such as 30 to 70 kDa, e.g., 30 to 65 kDa. FIGS. 1A and 1B provide illustrations of a single domain antibody/polymeric fluorescent tandem dye conjugate according to an embodiment of the invention. In the embodiment shown in FIG. 1A, the single domain antibody/polymeric fluorescent tandem dye conjugate includes a VhH nanobody conjugated to two polymeric fluorescent tandem dyes (each labeled “pendant chromophore”). FIG. 1B shows the same dye illustrated in FIG. 1A, with the structure of each of the polymeric fluorescent tandem dyes provided. As illustrated, each polymeric fluorescent tandem dye includes a peptide backbone having multiple pendant donor fluorophores and a pendant acceptor fluorophore, where fluorophores of the tandem dye are in energy transfer relationship. The different components of single domain antibody/polymeric fluorescent tandem dye conjugates of embodiments of the invention are now reviewed further in greater detail.

### Single Domain Antibody

As used herein, a single-domain antibody, which may also be referred to herein as a nanobody and may be designated sdAb, is an antibody fragment that includes a single monomeric variable antibody domain that is able to bind specifically to an antigen. In some instances, “single-domain antibody” or “sdAb” present in conjugates of the invention includes a single antigen-binding polypeptide having three complementary determining regions (CDRs). The sdAb alone is capable of specifically binding to a target antigen without pairing with a corresponding CDR-containing polypeptide.

A single domain antibody, i.e., nanobody, may include heavy chain variable domains or light chain variable domains. In some instances, a single domain antibody of embodiments of the present application includes a heavy chain variable domain. A single domain antibody may be derived from camelids (V_{H}H fragments) or cartilaginous fishes (V_{NAR} fragments). Alternatively, a single domain antibody may be derived from splitting the dimeric variable domains from IgG into monomers.

A single domain antibody comprises a variable region primarily responsible for antigen recognition and binding and a framework region. The “variable region,” also called the “complementarity determining region” (CDR), comprises loops which differ extensively in size and sequence based on antigen recognition. CDRs are generally responsible for the binding specificity of the nanobody. Distinct from the CDRs is the framework region. The framework region is relatively conserved and assists in overall protein structure. The framework region may comprise a large solvent-exposed surface consisting of a p-sheet and loop structure. A signal sequence, as known in the art, can be included, which is then cleaved from the mature nanobody.

A single domain antibody “which binds” an antigen of interest, is one that binds the antigen with sufficient affinity such that the single domain antibody or binding molecule is useful in targeting a desired antigen, and does not significantly cross-react with other proteins. In such embodiments, the extent of binding of the single domain antibody, i.e., nanobody, or other binding molecule to a non-targeted antigen will usually be no more than 10%, e.g., as determined by fluorescence activated cell sorting (FACS) analysis or radioimmunoprecipitation (RIA).

The term “variable” refers to the fact that certain portions of the variable domains differ extensively in sequence and are used in the binding and specificity of each particular variable domain for its particular antigen. However, the variability is not evenly distributed throughout the variable domains. It is concentrated in hypervariable regions. The more highly conserved portions of variable domains are called the framework regions (FRs). The term “hypervariable region” when used herein refers to the amino acid residues responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Immunoglobulin sequences, such as antibodies and antigen binding fragments derived there from (e.g., immunoglobulin single variable domains or ISVs) are used to specifically target the respective antigens disclosed herein. The generation of immunoglobulin single variable domains such as e.g., V_{HH}s or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

Unless indicated otherwise, the term “immunoglobulin single variable domain” or “ISV” is used as a general term to include, but not be limited to, antigen-binding domains or fragments such as V_{HH} domains or V_{H} or V_{L} domains, respectively. V_{HH} domains are of interest in embodiments of the present invention. The terms antigen-binding molecules or antigen-binding protein are used interchangeably and include also the term NANOBODIES®. The immunoglobulin single variable domains can be light chain variable domain sequences [e.g., a V_{L}-sequence), or heavy chain variable domain sequences (e.g., a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES™, including but not limited to V_{HH} sequences.

The immunoglobulin single variable domain includes immunoglobulin sequences of different origin, including, but not limited to, mouse, rat, rabbit, donkey, human, camelid, cartilaginous fish, e.g., shark, lamprey, immunoglobulin sequences, etc. The immunoglobulin single variable domain includes fully human, humanized, otherwise sequence optimized or chimeric immunoglobulin sequences. The immunoglobulin single variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. It is noted that the terms Nanobody or Nanobodies are registered trademarks of Ablynx N.V. and thus may also be referred to as NANOBODY® or NANOBODIES®, respectively.

An amino acid sequence such as, e.g., an immunoglobulin single variable domain or polypeptide according to the invention is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195.

Such immunoglobulin single variable domains may be derived in any suitable manner and from any suitable source, and may for example be naturally occurring V_{HH} sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VH Hs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Immunoglobulin single variable domains may comprise an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized immunoglobulin single variable domains of the invention can be obtained in any suitable manner known per se and thus are not strictly limited to polypeptides that have been obtained using a polypeptide that comprises a naturally occurring V_{HH} domain as a starting material.

Another class of immunoglobulin single variable domains of the invention comprises immunoglobulin single variable domains with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the description herein. Such “camelizing” substitutions may be inserted at amino acid positions that form and/or are present at the V_{H}-V_{L} interface, and/or at the so-called Camelidae hallmark residues (see for example WO 94/04678 and Davies and Riechmann (1994 and 1996)). The V_{H} sequence that is used as a starting material or starting point for generating or designing the camelized immunoglobulin single variable domains is preferably a V_{H} sequence from a mammal, more preferably the V_{H} sequence of a human being, such as a V_{H}3 sequence. However, it should be noted that such camelized immunoglobulin single variable domains of the invention can be obtained in any suitable manner known per se and thus are not strictly limited to polypeptides that have been obtained using a polypeptide that comprises a naturally occurring V_{H} domain as a starting material.

Single domain antibodies employed in embodiments of the invention may be engineered to provide for ease of manufacture. To facilitate the assembly of dye conjugates, a single domain antibody may be engineered to possess specific reactive sites for the covalent attachment of one or more polymeric tandem fluorescent dyes. By utilizing engineered reactive sites which are peripheral to the single domain antibody structure (rather than sites internal to the single domain antibody protein) the unique binding affinity of the single domain antibody is retained after dye conjugation. These reactive sites can be chosen and installed from a list of reactive groups commonly used for bioconjugation, where such groups include, but are not limited to, thiols, transcyclooctenes, and dibenzylcyclooctynes. Thiol groups allow site specific maleimide addition of polymeric fluorescent tandem dye to the single domain antibody. Transcyclooctene, or dibenzylcyclooctyne allow site specific polymeric fluorescent tandem dye attachment via strain promoted cycloaddition.

Single domain antibodies present in conjugates of the invention may vary in size, and in some instance have a molecular weight ranting from 10 to 25 kDa, such as 15 to 20 kDa, e.g., 15 to 17 kDa.

Single domain antibodies of conjugates of the invention may specifically bind to a variety of different targets. In some instances, the single domain antibodies specifically bind to cell surface proteins or cell markers, .e., a cell receptor and a cell surface antigen. In some cases, the single domain antibody may specifically bind to a cell surface antigen, where cell surfact antigens to which the single domain antibody may bind include, but are not limited to, CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71. In other embodiments, the single domain antibody may bind to an intracellular target. In such embodiments, intracellular targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, etc. Intracellular proteins of interest to which the single domain antibody may specifically bind include, but are not limited to: hormones, growth factors, transcription factors, receptors, enzymes, cytokines, osteo-inductive factors, colony stimulating factors, and the like. In some embodiments, single domain antibody specifically binds to an immunoglobulin, e.g., a primary antibody employed in a given assay, such as primary mouse or rate antibody, e.g., where the single antibody domain/polymeric tandem fluorescent dye is employed as a secondary label.

### Polymeric Fluorescent Tandem Dye

As summarized above, single domain antibody/polymeric fluorescent tandem dye conjugates of embodiments of the invention include a single domain antibody, e.g., as described above, conjugated (e.g., covalently bound to) one or more polymeric fluorescent tandem dyes. As reviewed above, in some instances conjugates include a single domain antibody conjugated to a plurality of polymeric tandem fluorescent dyes, e.g., 2 or more, such as 3 or more, including 4 or more, such as 5 or more polymer tandem fluorescent dyes. In some instances, the conjugates include 2 to 4 polymeric tandem fluorescent dyes, such as 2 to 3 polymeric tandem fluorescent dyes, e.g., 2 polymeric tandem fluorescent dyes.

Polymeric fluorescent tandem dyes found in conjugates of embodiments of the invention may vary. In some instances, polymeric fluorescent tandem dyes found in conjugates of the invention include: a polymeric backbone; a plurality of pendant donor fluorophores each independently linked to a repeat unit of the polymeric backbone; and one or more pendant acceptor fluorophores linked to a repeat unit of the polymeric backbone, where pendant donor and acceptor fluorophores are in energy transfer relationship. As such, polymeric tandem dyes of embodiments of the invention include one or more pendant donor fluorophores and one or more pendant acceptor fluorophores, configured in energy-receiving proximity to the one or more pendant donor fluorophores, e.g., where both are linked to a common polymeric backbone. In some embodiments, a plurality of pendant donor fluorophores are present and are configured in energy-transferring proximity to a pendant acceptor fluorophore(s), where in some instances the plurality of pendant donor fluorophores ranges from 2 to 20, such as 2 to 15, e.g., 2 to 10. The term “pendant group” refers to a sidechain group that is connected to the backbone but which is not part of the backbone itself. In embodiments of the polymeric tandem dyes, the donor fluorophore is capable of transferring energy to a linked acceptor fluorophore. As such, the subject tandem dyes include a linked acceptor signaling fluorophore in energy-receiving proximity to the donor fluorophore system, i.e., in energy-receiving proximity to at least one linked donor fluorophore. A particular configuration of pendant groups can be determined and controlled by the arrangement of the repeat units of the underlying polymeric backbone (also referred to herein as “modular scaffold” to which the pendant groups are attached. The dye conjugates can include a plurality of water solubilizing groups attached to the scaffold and/or the pendant groups at any convenient locations to provide a water soluble polymeric dye. The polymeric backbone, i.e., modular scaffold, can be composed of repeat units which form a polymeric backbone having sidechain groups to which the pendant groups can be attached. The repeat units can be arranged in a variety of configurations to provide for a tandem dye having desirable spectroscopic properties. The distances and arrangement between sites for covalent attachment of the pendant donor fluorophores and the acceptor fluorophore(s) (when present) can be controlled to provide for desirable energy transfer processes.

In some instances, the polymeric backbone is made up of non-conjugated repeat units having any convenient configuration, such as a linear, branched or dendrimer configuration. The polymeric backbone can be a linear polymer. The polymeric backbone can be branched. In some instances, the dye conjugate includes a plurality of pendant donor chromophore groups each independently linked to a non-conjugated repeat unit of the polymeric backbone. The configuration of pendant groups can be installed during or after synthesis of the polymeric backbone. The incorporation of pendant groups can be with achieved with a random configuration, a block configuration, or in a sequence-specific manner via stepwise synthesis, depending on the particular method of synthesis utilized.

The term “unit” refers to a structural subunit of a polymer. The term unit is meant to include monomers, co-monomers, co-blocks, repeating units, and the like. A “repeating unit” or “repeat unit” is a subunit of a polymer that is defined by the minimum number of distinct structural features that are required for the unit to be considered monomeric, such that when the unit is repeated n times, the resulting structure describes the polymer or a block thereof. In some cases, the polymer may include two or more different repeating units, e.g., when the polymer is a multiblock polymer, a random arrangement of units or a defined sequence, each block may define a distinct repeating unit. It is understood that a variety of arrangements of repeating units or blocks are possible and that in the depicted formula of the polymer backbones described herein any convenient linear arrangements of various lengths can be included within the structure of the overall polymer. It is understood that the polymer may also be represented by a formula in terms of mol % values of each unit in the polymer and that such formula may represent a variety of arrangements of repeat unit, such as random or multiblock polymer or a defined sequence of residues. In some cases, a repeating unit of the polymer includes a single monomer group. In certain instances, a repeating unit of the polymer includes two or more monomer groups, i.e., co-monomer groups, such as two, three, four or more co-monomer groups. The term “co-monomer” or “co-monomer group” refers to a structural unit of a polymer that may itself be part of a repeating unit of the polymer.

The polymeric backbone may have a random configuration of non-conjugated repeat units. The polymeric backbone may include a block or co-block configuration of non-conjugated repeat units. Alternatively, the polymeric backbone may include a particular defined sequence of non-conjugated repeat units, e.g., amino acid residues of a polypeptide sequence. These configurations can be characterized by polymeric segments of repeat units (e.g., as described herein), which segments can themselves be repeated throughout the modular scaffold.

By “non-conjugated” is meant that at least a portion of the repeat unit includes a saturated backbone group (e.g., a group having two or more consecutive single covalent bonds) which precludes pi conjugation or an extended delocalized electronic structure along the polymeric backbone from one repeat unit to the next. It is understood that even though one repeat unit may not be conjugated to an adjacent repeat unit, such a repeat unit may include one or more isolated unsaturated groups including an unsaturated bond (e.g., of an alkenylene group or an alkynylene group) and/or an aryl or heteroaryl group, which groups can be a part of the backbone. In some cases, each repeat unit of the polymeric backbone includes one sidechain including a linked pendant group or a chemo-selective tag for linking to a pendant group.

In certain embodiments of the tandem dyes, the polymeric backbone is a linear polymer. In certain cases, the linear polymer is selected from a peptide, a peptoid, a hydrocarbon polymer, and a PEG polymer. In certain cases, the linear polymer is a peptide. In certain cases, the linear polymer is a peptoid. In certain cases, the polymer is a hydrocarbon polymer. In certain other cases, the polymer is a PEG polymer. Further details regarding polymeric backbones that may be employed in embodiments of the invention are found in PCT application serial no. PCT/US2019/024662 published as WO2019/191482 and PCT application serial no. PCT/US2020/019510 published as WO2020/222894; the disclosures of which applications are herein incorporated by reference.

Tandem dyes of embodiments of the invention may include a linear polymer backbone of any number of units. As used herein the term “unit” refers to a structural subunit of a polymer. The term unit is meant to include monomers, co-monomers, co-blocks, segments, repeating units, and the like. A “repeating unit” is a subunit of a polymer that is defined by the minimum number of distinct structural features that are required for the unit to be considered monomeric, such that when the unit is repeated n times, the resulting structure describes the polymer or a block thereof. In some cases, the polymer may include two or more different repeating units, e.g., when the polymer is a multiblock polymer or a random arrangement of units, each block may define a distinct repeating unit, e.g., an n-block and a m-block. It is understood that a variety of arrangements of n and/or m repeating units or blocks are possible and that any convenient linear arrangements of co-blocks of various lengths are included within the structure of the overall polymer. It is understood that the polymer may also be represented by a formula in terms of mol % values of each unit in the polymer and that such formula may represent a variety of arrangements of repeat unit, such as random or multiblock polymer. In some cases, a repeating unit of the polymer includes a single monomer group. In certain instances, a repeating unit of the polymer includes two or more monomer groups, i.e., co-monomer groups, such as two, three, four or more co-monomer groups. As used herein, the term “co-monomer” or “co-monomer group” refers to a structural unit of a polymer that may itself be part of a repeating unit of the polymer.

In certain instances, the tandem dye includes a linear peptide backbone of from 2 to 100 amino acids, such as 2 to 90, 2 to 80, 2 to 70, 2 to 60, 2 to 50, 2 to or 2 to 30 amino acids. In some cases, the linear peptide backbone includes 2 or more amino acids, such as 5 or more, 10 or more, 15 or more, 20 or more, or more, 30 or more, up to a maximum of 100 amino acids. In certain cases, the tandem dye includes a linear peptide backbone of from 5 to 30 amino acids, such as 5 to 25, 5 to 20, 5 to 15, or 5 to 10 amino acids.

The backbone of the tandem dye may have any convenient length. In some cases, the particular number of monomeric repeating units or segments of the chromophore may fall within the range of 2 to 500,000, such as 2 to 100,000, 2 to 30,000, 2 to 10,000, 2 to 3,000 or 2 to 1,000 units or segments, or such as 5 to 100,000, 10 to 100,000, 100 to 100,000, 200 to 100,000, or 500 to 50,000 units or segments. In some instances, the particular number of monomeric repeating units or segments of the backbone may fall within the range of 2 to 1,000, such as 2 to 500, 2 to 100, 3 to 100, 4 to 100, 5 to 100, 6 to 100, 7 to 100, 8 to 100, 9 to 100 or 10 to 100 units or segments. In certain cases, the particular number of monomeric repeating units or segments of the backbone may fall within the range of 2 to 500, such as 2 to 400, 2 to 300, 2 to 200, or 2 to 100 units or segments. In certain cases, the particular number of monomeric repeating units or segments of the backbone may fall within the range of 2 to 100 repeating monomeric units, such as 2 to 90, 2 to 80, 2 to 70, 2 to 60, 2 to 50, 2 to 40, or 2 to 30 units or segments.

As summarized above, tandem dyes of embodiments of the invention include one or more pendant donor fluorophores linked to the polymeric backbone. Any convenient pendant donor fluorophore may be linked to the polymeric backbone, where donor fluorophores of interest include, but are not limited to: BODIPY fluorophores; aryl fluorophores, heteroaryl fluorophores, and the like. In some instances, the donor fluorophore is a BODIPY fluorophore.

The term “BODIPY fluorophore” refers to a pendant donor fluorophore of a tandem dye which includes a chromophore having the following boron-dipyrromethene (BODIPY) core structure:

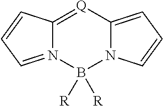

where Q is C or N and each R is any convenient boron substituent. In some cases, Q is C. In some instances, each R is independently selected from F, OH, H, alkyl, substituted alkyl, aryl, substituted aryl, heteroaryl, substituted heteroaryl, alkoxy, substituted alkoxy, alkynyl and substituted alkynyl. The BODIPY chromophore groups may be optionally further substituted, e.g., with a water solubilizing group and/or an aryl or heteroaryl substituent that imparts desirable light absorbing properties to the chromophore. Further description of BODIPY fluorophores that may be employed as donor fluorophores in embodiments of the invention are described in PCT application serial no. PCT/US2019/024662 published as WO 2019/191482; the disclosure of which is herein incorporated by reference. In some embodiments, donor fluorophores are fluorophores that include a aryl or heteroaryl chromophore group. Aryl or heteroaryl chromophore groups of interest which find use in embodiments of the invention include, but are not limited to, phenyl, biphenyl, benzooxazole, benzothiazole, poly-phenylene, and fused tricyclic groups, such as fluorene, carbazole, silole, biphenyl and bridged biphenyl. The aryl or heteroaryl chromophore groups may be optionally further substituted, e.g., with a water solubilizing group and/or an aryl or heteroaryl substituent that imparts desirable light absorbing properties to the aryl or heteraryl group. In some embodiments, donor fluorophores include a fused tricyclic aryl or heteroaryl. In some embodiments, donor fluorophores include one or more groups selected from fluorene, carbazole, silole, biphenyl and bridged biphenyl. A fused tricyclic chromophore is a group including a tricyclic aromatic group having three fused rings in a configuration where two aryl or heteroaryl 6-membered rings are fused to a central 5 or 6-membered carbocyclic or heterocyclic ring. In some cases, the fused tricyclic group includes two benzo or pyrido rings fused to a central 5 or 6 membered carbocyclic or heterocyclic ring. The fused tricyclic group can be linked to the sidechain of a co-monomer in the polymeric backbone via any convenient ring atoms of the fused rings. The central 5- or 6-membered ring may be a carbocycle or a heterocycle, aromatic or partially saturated, and may further include a sidechain substituent, e.g., a WSG and/or a linker to a chemoselective tag or the co-monomer sidechain. A bridged biphenyl co-monomer is a fused tricyclic group having a biphenyl group where the two phenyl rings are further linked with each other via a central 6 membered carbocyclic or heterocyclic ring. Further description of aryl/heteroaryl fluorophores that may be employed as donor fluorophores in embodiments of the invention are described in PCT application serial no. PCT/US2019/024662 published as WO 2019/191482; the disclosure of which is herein incorporated by reference. The number of repeat units of the donor water solvated light harvesting chromophore having linked acceptor signaling fluorophore groups may vary, where in some instances the number ranges from 1 mol % to 50 mol % of the repeat units, such as from 1 mol % to 25 mol %, 2 mol % to 25 mol %, 3 mol % to 25 mol %, 4 mol % to 25 mol %, 5 mol % to 25 mol % or from 10 mol % to mol %.

In addition to pendant donor fluorophore(s), tandem dyes of the invention include one or more acceptor fluorophores. Any convenient fluorescent dye may be utilized in the polymeric tandem dyes as an acceptor fluorophore. The acceptor fluorophore (e.g., each A) can be a small molecule fluorophore. The acceptor fluorophore (e.g., each A) can be a dye molecule selected from a rhodamine, a perylene, a diimide, a coumarin, a xanthene, a cyanine, a polymethine, a pyrene, a thiazine, an acridine, a dipyrromethene borondifluoride, a napthalimide, a phycobiliprotein, a peridinum chlorophyll protein, conjugates thereof, and combinations thereof. In certain embodiments, the acceptor fluorophore (A) is a cyanine dye, a xanthene dye, a coumarin dye, a thiazine dye or an acridine dye. In some instances, the acceptor fluorophore (A) is selected from DY 431, DY 485XL, DY 500XL, DY 610, DY 640, DY 654, DY 682, DY 700, DY 701, DY 704, DY 730, DY 731, DY 732, DY 734, DY 752, DY 778, DY 782, DY 800, DY 831, Biotium CF 555, Cy 3.5 and diethylamino coumarin. Fluorescent dyes of interest include, but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Lanthanide chelates of interest include, but are not limited to, europium chelates, terbium chelates and samarium chelates. In some embodiments, the polymeric tandem dye includes a multichromophore linked to an acceptor fluorophore selected from Cy3, Cy3.5, Cy5, Cy5.5, Cy7, Alexa488, Alexa 647 and Alexa700. In certain embodiments, the polymeric tandem dye includes a multichromophore linked to an acceptor fluorophore selected from Dyomics dyes (such as DY 431, DY 485XL, DY 500XL, DY 530, DY 610, DY 633, DY 640, DY 651, DY 654, DY 682, DY 700, DY 701, DY 704, DY 730, DY 731, DY 732, DY 734, DY 752, DY 754, DY 778, DY 782, DY 800 or DY 831), Biotium CF 555, Cy 3.5, and diethylamino coumarin. In certain cases, the acceptor fluorophore (A) is selected from fluorescein, 6-FAM, rhodamine, Texas Red, California Red, iFluor594, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight 350, DyLight 405, DyLight 488, DyLight 549, DyLight 594, DyLight 633, DyLight 649, DyLight 680, DyLight 750, DyLight 800, phycoerythrin, PerCP (peridinin chlorophyll-a Protein), PerCP-Cy5.5, JOE (6-carboxy-4′,5′-dichloro-2′,7′-dimelhoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY®530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, conjugates thereof and combinations thereof.

As mentioned above, where desired, the polymeric backbone and/or pendant fluorophores (i.e., donor and acceptor fluorophores) may include one or more water solubilizing groups (WSG). In some cases, the WSGs are pendant groups connected directly to the polymeric backbone, e.g., as sidechains of a polymeric backbone. In certain cases, the WSGs are substituent groups attached to a pendant donor fluorophore or pendant acceptor fluorophore. In some instances, each of the pendant donor flouorophore groups are substituted with one or more WSG. As used herein, the terms “water solubilizing group”, “water soluble group” and WSG are used interchangeably and refer to a group or substituent that is well solvated in aqueous environments e.g., under physiological conditions, and which imparts improved water solubility upon the molecule to which it is attached. A WSG can increase the solubility of a give polymeric tandem dye in a predominantly aqueous solution, as compared to a control dye which lacks the WSG. The water solubilizing groups may be any convenient hydrophilic group that is well solvated in aqueous environments. A water soluble tandem dye of the present disclosure has solubility under aqueous conditions that makes it especially suitable for application to a variety of biological assays. A variety of water soluble polymer groups can be adapted for use in the WSG of the subject dyes. Any convenient water solubilizing groups (WSG's) may be included in the dyes described herein to provide for increased water-solubility. While the increase in solubility may vary, in some instances the increase (as compared to the compound without the WSG(s)) is 2 fold or more, e.g., 5 fold, 10 fold, 25 fold, 50 fold, 100 fold or more. In some cases, the hydrophilic water solubilizing group is charged, e.g., positively or negatively charged. In certain cases, the hydrophilic water solubilizing group is a neutral hydrophilic group. In some embodiments, the WSG is branched (e.g., as described herein). In certain instances, the WSG is linear. In some embodiments, the WSG is a hydrophilic polymer, e.g., a polyethylene glycol, a modified PEG, a peptide sequence, a peptoid, a carbohydrate, an oxazoline, a polyol, a dendron, a dendritic polyglycerol, a cellulose, a chitosan, or a derivative thereof. Water solubilizing groups of interest include, but are not limited to, carboxylate, phosphonate, phosphate, sulfonate, sulfate, sulfinate, sulfonium, ester, polyethylene glycols (PEG) and modified PEGs, hydroxyl, amine, amino acid, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, —COOM′, —SO₃M′, —PO₃M′, —NR₃⁺, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from O, S, and NR^{ZZ}, and R^{ZZ} and R^{YY} are independently selected from H and C₁₋₃ alkyl. In some cases, a WSG is (CH₂)ₓ(OCH₂CH₂)_{y}OCH₃ where each x is independently an integer from 0-20, each y is independently an integer from 0 to 50. In some cases, the water solubilizing group includes a non-ionic polymer (e.g., a PEG polymer) substituted at the terminal with an ionic group (e.g., a sulfonate). Further details regarding suitable WSGs that may be present in dyes of embodiments of the invention may be found in PCT application serial no. PCT/US2019/024662 published as WO 2019/191482; the disclosure of which is herein incorporated by reference.

As summarized above, polymeric fluorescent tandem dyes of embodiments of the invention include a plurality of pendant donor fluorophores and one or more pendant acceptor fluorophores. In some instances, the number of donor fluorophores exceeds the number of acceptor fluorophores. In certain embodiments of the subject tandem dyes, the ratio of donor fluorophores to acceptor fluorophores is selected from 5:1, 6:1, 7:1, 8:1, 9:1, 10:1, and 20:2. In certain cases, the ratio of donor fluorophores to acceptor fluorophores is 5:1. In certain cases, the ratio of donor fluorophores to acceptor fluorophores is 6:1. In certain cases, the ratio of donor fluorophores to acceptor fluorophores is 7:1. In certain cases, the ratio of donor fluorophores to acceptor fluorophores is 8:1. In certain cases, the ratio of donor fluorophores to acceptor fluorophores is 9:1. In certain cases, the ratio of donor fluorophores to acceptor fluorophores is 10:1.

As mentioned above, in polymeric fluorescent tandem dyes of the invention, pendant donor and acceptor fluorophores are in energy transfer relationship. As such, in embodiments of the invention, excitation of the donor can lead to energy transfer to, and emission from, the covalently attached acceptor signaling fluorophore. Mechanisms for energy transfer between the donor chromophores to a linked acceptor signaling fluorophore include, for example, resonant energy transfer (e.g., Förster (or fluorescence) resonance energy transfer, FRET), quantum charge exchange (Dexter energy transfer) and the like. These energy transfer mechanisms can be relatively short range; that is, close proximity of chromophores of the light harvesting multichromophore system to each other and/or to an acceptor fluorophore provides for efficient energy transfer. Under conditions for efficient energy transfer, amplification of the emission from the acceptor fluorophore can occur where the emission from the luminescent acceptor fluorophore is more intense when the incident light (the “pump light”) is at a wavelength which is absorbed by, and transferred from, the chromophores of the light harvesting chromophore than when the luminescent acceptor fluorophore is directly excited by the pump light. By “efficient” energy transfer is meant 10% or more, such as 20% or more or 30% or more, 40% or more, 50% or more, of the energy harvested by the donor chromophores is transferred to the acceptor. By “amplification” is meant that the signal from the acceptor fluorophore is 1.5× or greater when excited by energy transfer from the donor light harvesting chromophore system as compared to direct excitation of the acceptor fluorophore with incident light of an equivalent intensity. The signal may be measured using any convenient method. In some cases, the 1.5× or greater signal refers to an intensity of emitted light. In certain cases, the 1.5× or greater signal refers to an increased signal to noise ratio. In certain embodiments of the tandem dye, the acceptor fluorophore emission is 1.5 fold greater or more when excited by the chromophore as compared to direct excitation of the acceptor fluorophore with incident light, such as 2-fold or greater, 3-fold or greater, 4-fold or greater, 5-fold or greater, 6-fold or greater, 8-fold or greater, 10-fold or greater, 20-fold or greater, 50-fold or greater, 100-fold or greater, or even greater as compared to direct excitation of the acceptor fluorophore with incident light.

The tandem dyes of embodiments of the invention may be of any convenient molecular weight (MW). In some cases, the MW of the tandem dye may be expressed as an average molecular weight. In some instances, the tandem dye has an average molecular weight in the range of 500 to 500,000, such as from 1,000 to 100,000, from 2,000 to 100,000, from 10,000 to 100,000 or even an average molecular weight in the range of 50,000 to 100,000 daltons. In some instances, the polymeric fluorescent dyes have a molecular weight ranging from 5 to 75 kDa, such as 10 to 50 kDa, such as 15 to 45 kDa. In certain embodiments, the molecular weight of the polymeric fluorescent dye ranges from 1 to 30 kDa, such as 2 to 25 kDa, including 2 to 20 kDa.

In some instances, the tandem dye exhibits an effective Stokes shift of 100 nm or more, such as 110 nm or more, 120 nm or more, 130 nm or more, 140 nm or more, 150 nm or more, 160 nm or more, 170 nm or more, 180 nm or more, 190 nm or more, 200 nm or more, 250 nm or more when the light harvesting chromophore is directly excited with incident light. In some cases, the effective Stokes shift of the tandem dye is up to about 300 nm such as 100-300 nm, 100-250 nm or 100-200 nm.

The emission of the polymeric tandem dye can have a quantum yield of 0.03 or more, such as a quantum yield of 0.04 or more, 0.05 or more, 0.06 or more, 0.07 or more, 0.08 or more, 0.09 or more, 0.1 or more, 0.15 or more, 0.2 or more, 0.3 or more or even more. In some instances, the polymeric tandem dye has an extinction coefficient of 5×10⁵ cm⁻¹M⁻¹ or more, such as 6×10⁵ cm⁻¹M⁻¹ or more, 7×10⁵ cm⁻¹M⁻¹ or more, 8×10⁵ cm⁻¹M⁻¹ or more, 9×10⁵ cm⁻¹M⁻¹ or more, such as 1×10⁶ cm⁻¹M⁻¹ or more, 1.5×10⁶ cm⁻¹M⁻¹ or more, 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In some embodiments, the polymeric tandem dye has a molar extinction coefficient of 5×10⁵ M⁻¹cm⁻¹ or more. In certain embodiments, the tandem dye has a molar extinction coefficient of 1×10⁶ M⁻¹cm⁻¹ or more.

In embodiments, the subject tandem dyes provide for fluorescence emissions from acceptor fluorophores that are brighter than the emissions which are possible from such fluorescent dyes in isolation. The emission of the polymeric tandem dye can have a brightness of 50 mM⁻¹cm⁻¹ or more, such as 60 mM⁻¹cm⁻¹ or more, 70 mM⁻¹cm⁻¹ or more, 80 mM⁻¹cm⁻¹ or more, 90 mM⁻¹cm⁻¹ or more, 100 mM⁻¹cm⁻¹ or more, 150 mM⁻¹cm⁻¹ or more, 200 mM⁻¹cm⁻¹ or more, 250 mM⁻¹cm⁻¹ or more, 300 mM⁻¹cm⁻¹ or more, or even more. In certain instances, the emission of the tandem dye has a brightness that is at least 5-fold greater than the brightness of a directly excited acceptor fluorophore, such as at least 10-fold greater, at least 20-fold greater, at least 30-fold greater, at least 50-fold greater, at least 100-fold greater, at least 300-fold greater, or even greater than the brightness of a directly excited acceptor fluorophore.

In addition to attributes such as described above, tandem dyes of embodiments of the invention may have one or more additional desirable spectroscopic properties, such as a particular emission maximum wavelength, extinction coefficient, quantum yield, and the like. As discussed above, the subject tandem dyes provide, in some instances, for a donor chromophore having an absorption maxima of from 555 nm to 585 nm, such as 555 nm to 575 nm, such as 555 to 575 nm, such as 555 to 574 nm, 555 to 573 nm, 555 to 572 nm, 555 to 571 nm, 555 to 570 nm, 555 to 569 nm, 555 to 568 nm, 555 to 567 nm, 555 to 566 nm, 555 to 565 nm, 555 to 564 nm, 555 to 563 nm, or 555 to 562 nm.

A variety of emission profiles which depend on a variety of factors such as the selected co-monomers, linking groups, substituents and linked acceptor fluorophores of which the tandem dyes are composed. In some embodiments, the chromophore has an emission maximum wavelength in the range of 300 to 900 nm, such as 350 to 850 nm, 350 to 600 nm, 360 to 500 nm, 370 to 500 nm, 380 to 500 nm, 390 to 500 nm or 400 to 500 nm, where specific examples of emission maxima of interest include, but are not limited to: 395 nm±5 nm, 460 nm±5 nm, 490 nm±5 nm, 550 nm±5 nm, 560 nm±5 nm, 605 nm±5 nm, 650 nm±5 nm, 680 nm±5 nm, 700 nm±5 nm, 805 nm±5 nm. In certain instances, the chromophore has an emission maximum wavelength selected from the group consisting of 395 nm, 460 nm, 490 nm, 550 nm, 560 nm, 605 nm, 650 nm, 680 nm, 700 nm and 805 nm. In certain instances, the tandem dye has an emission maximum wavelength of 395 nm±5 nm. In some instances, the tandem dye itself has an emission maximum wavelength in the range of 375 to 900 nm (such as in the range of 380 nm to 900 nm, 390 nm to 900 nm, or 400 nm to 900 nm).

In some instances, the tandem dye has an extinction coefficient of 5×10⁵ cm⁻¹M⁻¹ or more, such as 6×10⁵ cm⁻¹M⁻¹ or more, 7×10⁵ cm⁻¹M⁻¹ or more, 8×10⁵ cm⁻¹M⁻¹ or more, 9×10⁵ cm⁻¹M⁻¹ or more, such as 1×10⁶ cm⁻¹M⁻¹ or more, 1.5×10⁶ cm⁻¹M⁻¹ or more, 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In such cases, the tandem may have 5 or more repeating units, such as 6 or more, 7 or more, 8 or more, 9 or more, 10 or more, or even more repeating units. In some embodiments, the tandem dye has a molar extinction coefficient of 5×10⁵ M⁻¹cm⁻¹ or more. In certain embodiments, the chromophore has a molar extinction coefficient of 1×10⁶ M⁻¹cm⁻¹ or more.

In some instances, the tandem dye has an extinction coefficient of 40,000 cm⁻¹M⁻¹ per repeating unit or more, such as 45,000 cm⁻¹M⁻¹ per repeating unit or more, 50,000 cm⁻¹M⁻¹ per repeating unit or more, 55,000 cm⁻¹M⁻¹ per repeating unit or more, 60,000 cm⁻¹M⁻¹ per repeating unit or more, 70,000 cm⁻¹M⁻¹ per repeating unit or more, 80,000 cm⁻¹M⁻¹ per repeating unit or more, 90,000 cm⁻¹M⁻¹ per repeating unit or more, 100,000 cm⁻¹M⁻¹ per repeating unit or more, or even more. In some instances, the 40,000 cm⁻¹M⁻¹ per repeating unit or more described herein is an average extinction coefficient. In certain instances, the repeat unit of the chromophore may include a single monomer, two co-monomers, or three or more co-monomers. In some instances, the chromophore has an extinction coefficient of 40,000 cm⁻¹M⁻¹ per co-monomer or more, such as 45,000 cm⁻¹M⁻¹ per co-monomer or more, 50,000 cm⁻¹M⁻¹ per co-monomer or more, 55,000 cm⁻¹M⁻¹ per co-monomer or more, 60,000 cm⁻¹M⁻¹ per co-monomer or more, 70,000 cm⁻¹M⁻¹ per co-monomer or more, 80,000 cm⁻¹M⁻¹ per co-monomer or more, 90,000 cm⁻¹M⁻¹ per co-monomer or more, 100,000 cm⁻¹M⁻¹ per co-monomer or more, or even more. In some instances, the 40,000 cm⁻¹M⁻¹ per co-monomer or more is an average extinction coefficient.

In some embodiments, the polymeric tandem dyes can include a segment of the formula (Ia):

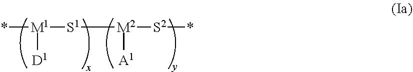

wherein:
each M¹ and M² is independently an unsaturated co-monomer (e.g., an amino acid residue);
each S¹ and S² is independently a non-conjugated spacer unit;
each D¹ is independently a pendant donor fluorophore (e.g., as described herein) linked to M¹;
each A¹ is independently an acceptor fluorophore linked to M²;
x is 75 mol % or more; and
y is 25 mol % or less.
   The first (M¹-S¹) and second repeat units (M²-S²) can be arranged in a random or co-block configuration. In certain cases of formula (Ia), the D¹ pendant groups of the first repeat units include two or more (e.g., two or three) distinct types of pendant light absorbing chromophores that together provide a light harvesting multichromophore system. In certain instances of formula (Ia), the D¹ pendant groups of the first repeat units are all the same.

In some instances of formula (Ia), x is 80 mol % or more, such as 85 mol % or more, 90 mol % or more, 95 mol % or more, 96 mol % or more, 97 mol % or more, 98 mol % or more, or 99 mol % or more. In some instances of formula (Ia), y is 20 mol % or less, such as 15 mol % or less, 10 mol % or less, 5 mol % or less, 4 mol % or less, 3 mol % or less, 2 mol % or less, 1 mol % or less.

In some instances, the polymeric tandem dye includes a segment of formula (IIa):

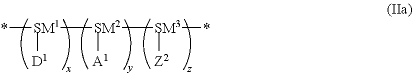

wherein:
the polymeric backbone of non-conjugated repeat units comprises SM¹, SM² and SM³ co-monomers that are each independently a non-conjugated co-monomer;
each D¹ is independently a pendant donor chromophore linked to SM¹;
each A¹ is independently an acceptor fluorophore linked to SM²;
each Z² is an optional sidechain group linked to SM³;
x is 50 mol % or more; and
y+z is 50 mol % or less.

Z² can be absent or any convenient sidechain group, such as a light absorbing chromophore, a chemoselective tag, a linker, a linked biomolecule, a acceptor fluorophore, a WSG (e.g., as described in PCT Application Serial No. PCT/US2019/024662 published as WO 2019/191482, the disclosure of which is herein incorporated by reference), etc. In certain cases of formula (IIa), SM³ is a spacer co-monomer where Z² is absent. In certain instances of formula (IIa), SM³ is a co-monomer including a Z² group that is a second pendant light absorbing chromophore, where each D¹ and each Z² together provide a light harvesting multichromophore system. In some cases, SM³ is a co-monomer including a second chemoselective tag (Z²), e.g., a protected functional group or a tag that is orthogonal to Z¹ that provides for the selective installation of a moiety of interest.

In certain cases of formula (IIa), x is 60 mol % or more, such as 65 mol % or more, 70 mol % or more, 75 mol % or more, 80 mol % or more, 85 mol % or more, 90 mol % or more, 95 mol % or more, or even more. In certain instances of formula (IIa), y+z is 40 mol % or less, such as 30 mol % or less, 25 mol % or less, 20 mol % or less, 15 mol % or less, 10 mol % or less, 5 mol % or less, or even less. In certain instances of formula (IIa), y is at least 1 mol % and 25 mol % or less, such as 20 mol % or less, 15 mol % or less, 10 mol % or less, 5 mol % or less, or even less. In certain instances of formula (IIa), z is at least 1 mol % and 10 mol % or less, such as 5 mol % or less, or even less.

In some instances, the polymeric tandem dye includes a segment of formula (IIIa):

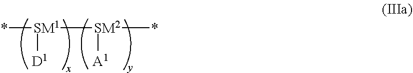

wherein:
the polymeric backbone of non-conjugated repeat units comprises SM¹ and SM² co-monomers that are each independently a non-conjugated co-monomer;
each D¹ is independently a pendant donor chromophore linked to SM¹;
each A¹ is independently an acceptor fluorophore linked to SM²;
x is 75 mol % or more; and
y is 25 mol % or less.

In certain embodiments of formula (IIIa), SM¹ and SM² are each independently a saturated non-conjugated co-monomer, e.g., a co-monomer providing only single covalent C—C bonds. In some embodiments of formula (IIIa), SM¹ and SM² are each independently a partially saturated non-conjugated co-monomer, e.g., a co-monomer providing an isolated double C═C covalent bond in a backbone of saturated covalent bonds. The first and second repeat units (SM¹ and SM²) of formula (IIIa) can be arranged in a random configuration, a block or co-block configuration, or in a particular sequence. In certain cases of formula (IIIa), the D¹ pendant groups of the SM¹ include two or more (e.g., two or three) distinct types of pendant light absorbing chromophores that together provide a light harvesting multichromophore system. In certain instances of formula (IIIa), the D¹ pendant groups of the first repeat units are all the same.

In some instances of formula (IIIa), x is 80 mol % or more, such as 85 mol % or more, 90 mol % or more, 95 mol % or more, 96 mol % or more, 97 mol % or more, 98 mol % or more, or 99 mol % or more. In some instances of formula (IIIa), y is 20 mol % or less, such as 15 mol % or less, 10 mol % or less, 5 mol % or less, 4 mol % or less, 3 mol % or less, 2 mol % or less, 1 mol % or less.

In certain instances, the polymeric tandem dye is of formula (IVa):

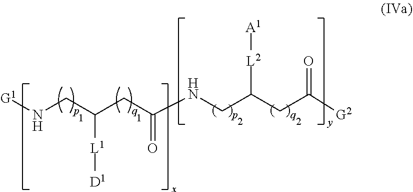

wherein:
each D¹ is independently a pendant donor chromophore group;
each A¹ is independently an acceptor fluorophore;
each L¹ and L² are independently a linker;
p₁ and q₁ are independently 0 or 1 wherein p₁+q₁≤1;
p₂ and q₂ are independently 0 or 1 wherein p₁+q₁≤1;
x is 75 mol % or more;
y is 25 mol % or less; and
G¹ and G² are each independently selected from a terminal group, a polymer segment, a light absorbing (e.g., donor) chromophore group, an acceptor fluorophore, a linker and a linker to a single domain antibody, e.g., as described above.

In some embodiments of formula (IVa), p₁ and p₂ are each 0 and q₁ and q₂ are each 1 (e.g., 33-amino acid residues). In some embodiments of formula (IVa), p₁ and p₂ are each 1 and q₁ and q₂ are each 0 (e.g., 32-amino acid residues). In some cases,_p₁, p₂, q₁ and q₂ are each 0 and the polymeric tandem dye is of formula (Va):

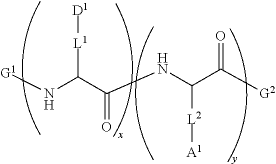

wherein:
each D¹ is independently a pendant donor chromophore group;
each A¹ is independently an acceptor fluorophore;
L¹ and L² are each independently a linker;
x is 75 mol % or more;
y is 25 mol % or less; and
G¹ and G² are each independently selected from a terminal group, a polymer segment, a light absorbing (e.g., donor) chromophore group, a linker and a linker to a single domain antibody, e.g., as described above. It is understood that the tandem dyes described by formula (Va) include any convenient arrangements of co-monomers in a defined linear sequence, which have in total the defined mol % ratios of x and y. In some cases, the A¹ containing co-monomers are spaced throughout the sequence of the polymeric backbone and as such are always flanked on both sides by one or more D1 containing co-monomers.

In certain instances of formula (Va), the polymeric tandem dye includes a segment of formula (VIa):

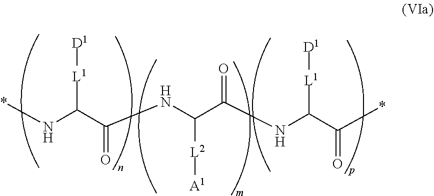

wherein:
each D¹ is independently a pendant donor chromophore group;
each A¹ is independently an acceptor chromophore;
each L¹ and L² are independently a linker;
n and p are each independently an integer from 1 to 20 wherein n+p≥2; and
m is 1 or 2.
   In some cases of formula (VIa), n and p are each independently 1 to 10 such as 2 to 20, 3 to 10 or 3 to 6. In some instances of formula (VIa), n+p is an integer from 2 to 20, such as 3 to 20, 4 to 20, 5 to 20, 5 to 15 or 5 to 12. In certain embodiments of formula (VIa), m is 1.

The subject polymeric tandem dyes can include multiple segments of formula (VIa) where each segment includes one isolated A¹ containing co-monomers flanked by blocks of D¹ containing co-monomers. In some cases, the multichromophore includes two or more segments of formula (VIa) located directed adjacent to each other to provide two isolated A¹ containing co-monomers separated by a block of 2-20 D¹ containing co-monomers, such as a block of 3 to 20, 4 to 20, 5 to 20, 5 to 15 or 5 to 12 D¹ containing co-monomers. As such, in certain embodiments, the polymeric tandem dye includes q segments of a block copolymer and is of formula (VIIa):

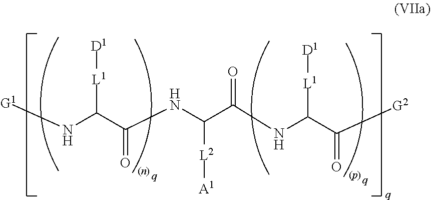

wherein: each (n)_{q} and each (p)_{q} is independently an integer from 1 to 20, wherein for each of the q segments (n)_{q}+(p)_{q}≥3; and q is an integer from 1 to 100.

In certain embodiments, the polymeric tandem dye has the formula (VIIIa):

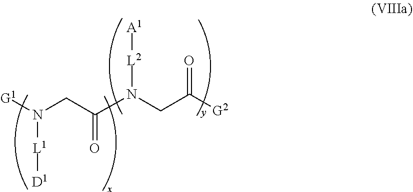

wherein
each D¹ is independently a pendant donor chromophore group;
each A¹ is independently an acceptor chromophore;
each L¹ and L² is independently a linker;
x is 75 mol % or more;
y is 25 mol % or less; and
G¹ and G² are each independently selected from a terminal group, a polymer segment, a donor chromophore group, a linker and a linker to a single domain antibody, e.g., as described above.

In certain embodiments, the polymeric tandem dye has the formula (IXa):

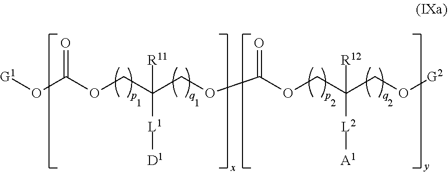

wherein:
each D¹ is independently a pendant BODIPY donor chromophore;
each A¹ is independently an acceptor fluorophore;
each L¹ and L² is independently a linker;
x is 75 mol % or more;
y is 25 mol % or less; and
G¹ and G² are each independently selected from the group consisting of a terminal group, a polymer segment, a donor chromophore group, an acceptor fluorophore, a linker and a linker to a single domain antibody, e.g., as describe above.

In some instances of formulae (IVa), (Va), (XIIIa) and (IXa), x is 80 mol % or more, such as 85 mol % or more, 90 mol % or more, 95 mol % or more, 96 mol % or more, 97 mol % or more, 98 mol % or more, or 99 mol % or more. In some instances of formula (IV), (V), (XIII) and (IX), y is 20 mol % or less, such as 15 mol % or less, 10 mol % or less, mol % or less, 4 mol % or less, 3 mol % or less, 2 mol % or less, 1 mol % or less.

In certain embodiments, the polymeric tandem dye has the formula (Xa):

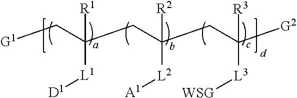

wherein:
each D¹ is independently a pendant donor chromophore;
each A¹ is independently an acceptor fluorophore;
each L¹, L² and L³ is independently a linker;
a, b and c are mol % values for each co-monomer;
d represents the total polymerization or average length of the polymer (e.g., d is 2-1000, such as 2-500, 2-200, 2-100 or 2-50);
WSG is a water solubilizing group (e.g., as described in PCT application serial no. PCT/US2019/024662 published as WO 2019/191482, the disclosure of which is herein incorporated by reference); and
G¹ and G² are each independently selected from the group consisting of a terminal group, a polymer segment, a donor chromophore group, an acceptor fluorophore, a linker and a linker to a single domain antibody, e.g., as described above.

In some instances of formula (Xa), c=0. In some instances of formula (Xa), a>0 and b>0. In some instances of formula (Xa), a is 80 mol % or more, such as 85 mol % or more, 90 mol % or more, 95 mol % or more, 96 mol % or more, 97 mol % or more, 98 mol % or more, or 99 mol % or more. In some instances of formula (Xa), b is 20 mol % or less, such as 15 mol % or less, 10 mol % or less, 5 mol % or less, 4 mol % or less, 3 mol % or less, 2 mol % or less, 1 mol % or less. In some instances of formula (Xa), a is 65-95 mol %, b is 5-35 mol % and c is 0-30 mol %, where a+b+c=100%.

Any convenient end groups (e.g., G¹ and G²) may be utilized at the terminals of the tandem fluorescent dyes. As used herein, the terms “end group” and “terminal group” are used interchangeably to refer to the groups located at the terminals of the polymeric structure of the light harvesting chromophore, e.g., as described herein. G¹ and G² groups of interest include, but are not limited to a terminal capping group, a π conjugated segment, a linker and a linker to a single domain antibody, e.g., as described above. In some embodiments, a terminal capping groups is a monovalent group which is conjugated to the backbone of the light harvesting chromophore after polymerization. In certain instances, the terminal capping group is an aryl, a substituted aryl, a heteroaryl, a substituted heteroaryl, an alkyl or a substituted alkyl. In some cases, the terminal co-monomer is directed linked to a chemoselective tag or linker. In certain cases, the terminal capping group is derived from a monomer used in the method of polymerization, e.g., a terminal group such as a halogen (e.g., Br), a boronic acid or a boronic ester, which is capable of undergoing further conjugation. In some instances, G¹ and/or G² is a π conjugated segment. As used herein, a n conjugated segment refers to any convenient segment of a conjugated polymer to which the light harvesting chromophore may be conjugated, i.e., allowing delocalization of pi electron across adjacent units. In certain embodiments, G¹ and/or G² is a linker, such as a linker including a functional group suitable for conjugation to a specific binding moiety. It is understood that linkers located at the G¹ and/or G² positions of the light harvesting chromophore may be selected so as to be orthogonal to any other linkers including chemoselective tags (e.g., as described herein) that may be present at a sidechain of the light harvesting chromophore. In certain embodiments, an amino functional group or derivative thereof is included at G¹ and/or G². In certain embodiments, a carboxylic acid functional group or derivative thereof is included at G¹ and/or G².

In some embodiments of the formulae described herein, at least one of G¹ and G² is -L⁶-Z⁴ where L⁶ is a linker (e.g., as described herein) and Z⁴ is a specific binding member (e.g., as described herein). In some embodiments of formulae described herein, at least one of G¹ and G² is -L⁶-Z³ where L⁶ is a linker (e.g., as described herein) and Z³ is a chemoselective tag (e.g., as described herein). Any convenient chemoselective tag and conjugation chemistries can be adapted for use in the subject light harvesting chromophores. Chemoselective tags of interest include, but are not limited to, amine, active ester, maleimide, thiol, sulfur(VI) fluoride exchange chemistry (SuFEX), sulfonyl fluoride, Diers Alder cycloaddition click reagents and click chemistry, tetrazine, transcyclooctene, aldehyde, alkoxylamine, alkynes, cyclooctynes, azide, and the like. In some instances, Z³ is selected from the group consisting of carboxylic acid, active ester (e.g., N-hydroxy succinimidyl ester (NHS) or sulfo-NHS), amino, maleimide, iodoacetyl and thiol. In certain embodiments of formulae described herein, at least one of G¹ and G² is described by the following structure:

*—Ar-L-Z

where Ar is a π-conjugated aryl group, L is a linker and Z is a chemoselective tag or a specific binding member. In some cases, the L-Z group can be connected directed to a terminal co-monomer. In certain embodiments of formulae described herein, at least one of G¹ and G² is described by the following structure:

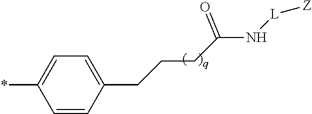

wherein:
q is 0 or an integer from 1-12;
L is an optional linker; and
Z is a chemoselective tag or a specific binding member. Further details regarding WSG groups that may be found in tandem dyes of embodiments of the invention are found in PCT application serial no. PCT/US2019/024662 published as WO 2019/191482, the disclosure of which is herein incorporated by reference

### Representative Single Domain Antibody/Polymeric Fluorescent Tandem Dye Conjugate

FIGS. 1A and 1B provide views of a single domain antibody/polymeric fluorescent tandem dye conjugate according to an embodiment of the invention. In FIG. 1A, the illustrated single domain antibody/polymeric fluorescent tandem dye conjugate includes a V_{H}H single domain antibody conjugated to two polymer fluorescent dyes (labeled “Tandem ‘Cobalt’ Dye”). The tandem dyes may be conjugated to any convenient residue of the single domain antibody, where residues of interest for conjugation or linking are residues which, when linked to the dye, will not adversely impact the binding activity of the single domain antibody. Residues that may be employed for conjugation to dyes may be naturally occurring or residues that are engineered into the single domain antibody, as desired, such as terminal cysteines or unnatural amino acids. FIG. 1B provides a more detailed view of the conjugate illustrated in FIG. 1A, showing the structure of the polymeric fluorescent tandem dyes as well as the PEG water solubilizing linkers that link the dyes to V_{H}H single domain antibody. While the length of the linker may vary, in some instance the linker ranges from 10 to 50 nm, such as 15 to 40 nm, e.g., 20 to 30 nm. A given linker may be flexible, e.g., PEG, or rigid, e.g., poly-glycine chain in place of PEG for example, as desired

### Methods

Aspects of the invention include methods of evaluating a sample for the presence of a target analyte. Aspects of the methods include contacting a sample with a single domain antibody/polymeric fluorescent dye conjugate that specifically binds the target analyte to produce an assay composition comprising the single domain antibody/polymeric fluorescent dye conjugate contacted sample. In the following section, the target analyte may be a target molecule of interest or reagent, e.g., primary antibody, bound to the target molecule, depending on whether the single domain antibody/polymeric fluorescent dye conjugate is employed as a primary or secondary label. Any convenient method may be used to contact the sample with a single domain antibody/polymeric fluorescent tandem dye conjugate that specifically binds to the target analyte to produce the assay composition. In some instances, the sample is contacted with the single domain antibody/polymeric fluorescent tandem dye conjugate under conditions in which the single domain antibody component of the conjugate specifically binds to the target analyte, if present. For specific binding of the single domain antibody of the conjugate with the target analyte, an appropriate solution may be used that maintains the biological activity of the components of the sample and the signal domain antibody. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. Various media are commercially available and may be used according to the nature of the target analyte, including dMEM, HBSS, dPBS, RPMI, Iscove's medium, etc., in some cases supplemented with fetal calf serum or human platelet lysate. The final components of the solution may be selected depending on the components of the sample which are included. The temperature at which specific binding of the singled domain antibody of the conjugate to the target analyte takes place may vary, and in some instances may range from 5° C. to 50° C., such as from 10° C. to 40° C., 15° C. to 40° C., 20° C. to 40° C., e.g., 20° C., 25° C., 30° C., 35° C. or 37° C. (e.g., as described above). In some instances, the temperature at which specific binding takes place is selected to be compatible with the biological activity of the single domain antibody and/or the target analyte. In certain instances, the temperature is 25° C., 30° C., 35° C. or 37° C. In certain cases, the temperature at which specific binding takes place is room temperature (e.g., 25° C.), 30° C., 35° C. or 37° C. Any convenient incubation time for specific binding may be selected to allow for the formation of a desirable amount of binding complex, and in some instances, may be 1 minute (min) or more, such as 2 min or more, 10 min or more, 30 min or more, 1 hour or more, 2 hours or more, or even 6 hours or more.

Any convenient single domain antibodies may be utilized in the conjugates employed in methods of the invention. Single domain antibodies of interest include, but are not limited to, those single domain antibodies that specifically bind cell surface proteins of a variety of cell types, including but not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that have a convenient cell surface marker or antigen that may be captured by a convenient specific binding member conjugate. In some embodiments, the target cell is selected from HIV containing cell, a Treg cell, an antigen-specific T-cell populations, tumor cells or hematopoetic progenitor cells (CD34+) from whole blood, bone marrow or cord blood. Any convenient cell surface proteins or cell markers may be targeted for specific binding to the conjugates employed in the subject methods. In some embodiments, the target cell includes a cell surface marker selected from a cell receptor and a cell surface antigen. In some cases, the target cell may include a cell surface antigen such as CD11 b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71.

Any convenient targets may be selected for evaluation utilizing the subject methods. Targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, transcription factor, receptors, enzymes, cytokines, osteo-inductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the polymeric dye conjugates include an antibody or antibody fragment. Any convenient target analyte that specifically binds an antibody or antibody fragment of interest may be targeted in the subject methods.

In some embodiments, the target analyte is associated with a cell. In certain instances, the target analyte is a cell surface marker of the cell. In certain cases, the cell surface marker is selected from the group consisting of a cell receptor and a cell surface antigen. In some instances, the target analyte is an intracellular target, and the method further includes permeabilizing or lysing the cell. As such, a single domain antibody of a given conjugate employed in methods of the invention may target a cell surface or intracellular antigen. Alternatively, a single domain antibody of a given conjugate employed in methods of the invention may target a primary antibody that in turn specifically binds to a target cell surface or intracellular antigen.

In some embodiments, the sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral cells (e.g., HIV).

The single domain antibody/polymeric fluorescent tandem dye conjugates find use in the subject methods, e.g., for labeling a target cell, particle, target or analyte with a polymeric tandem fluorescent dye. For example, single domain antibody/polymeric fluorescent tandem dye conjugates find use in labeling cells to be processed (e.g., detected, analyzed, and/or sorted) in a flow cytometer. The single domain antibody/polymeric fluorescent tandem dye conjugates may include single domain antibodies that specifically bind to, e.g., cell surface proteins of a variety of cell types (e.g., as described herein). The labelled single domain antibody/polymeric fluorescent tandem dye conjugates may be used to investigate a variety of biological (e.g., cellular) properties or processes such as cell cycle, cell proliferation, cell differentiation, DNA repair, T cell signaling, apoptosis, cell surface protein expression and/or presentation, and so forth. Labelled specific binding members may be used in any application that includes (or may include) antibody-mediated labeling of a cell, particle or analyte.

Aspects of the methods include assaying the assay composition, i.e., single domain antibody/polymeric fluorescent tandem dye conjugate contacted sample, for the presence of a single domain antibody/polymeric fluorescent tandem dye conjugate-target analyte binding complex to evaluate whether the target analyte is present in the sample. Once the sample has been contacted with the single domain antibody/polymeric fluorescent tandem dye conjugate, any convenient method may be utilized in assaying the assay composition that is produced for the presence of a single domain antibody/polymeric fluorescent tandem dye conjugate-target analyte binding complex. The single domain antibody/polymeric fluorescent tandem dye conjugate-target analyte binding complex is the binding complex that is produced upon specific binding of the specific binding member of the conjugate to the target analyte (or primary binding member, e.g., primary antibody, to the target antigen depending on the embodiment), if present. Assaying the assay composition may include detecting a fluorescent signal from the binding complex, if present. In some cases, the assaying includes a separating step where the target analyte, if present, is separated from the sample. A variety of methods can be utilized to separate a target analyte from a sample, e.g., via immobilization on a support. Assay methods of interest include, but are not limited to, any convenient methods and assay formats where pairs of specific binding members such as avidin-biotin or hapten-anti-hapten antibodies find use, are of interest. Methods and assay formats of interest that may be adapted for use with the subject compositions include, but are not limited to, flow cytometry methods, in-situ hybridization methods, enzyme-linked immunosorbent assays (ELISAs), western blot analysis, magnetic cell separation assays and fluorochrome purification chromatography.

In certain embodiments, the method further includes contacting the sample with a second specific binding member that specifically binds the target analyte. In certain instances, the second specific binding member is support bound. Any convenient supports may be utilized to immobilize a component of the subject methods (e.g., a second specific binding member). In certain instances, the support is a particle, such as a magnetic particle. In some instances, the second specific binding member and the polymeric dye conjugate produce a sandwich complex that may be isolated and detected, if present, using any convenient methods. In some embodiments, the method further includes flow cytometrically analyzing the polymeric dye conjugate-target analyte binding complex, i.e., a fluorescently labelled target analyte. Assaying for the presence of a polymeric dye conjugate-target analyte binding complex may provide assay results (e.g., qualitative or quantitative assay data) which can be used to evaluate whether the target analyte is present in the sample.

Any convenient supports may be utilized in the subject methods to immobilize any convenient component of the methods, e.g., labelled specific binding member, target, secondary specific binding member, etc. Supports of interest include, but are not limited to: solid substrates, where the substrate can have a variety of configurations, e.g., a sheet, bead, or other structure, such as a plate with wells; beads, polymers, particle, a fibrous mesh, hydrogels, porous matrix, a pin, a microarray surface, a chromatography support, and the like. In some instances, the support is selected from the group consisting of a particle, a planar solid substrate, a fibrous mesh, a hydrogel, a porous matrix, a pin, a microarray surface and a chromatography support. The support may be incorporated into a system that it provides for cell isolation assisted by any convenient methods, such as a manually-operated syringe, a centrifuge or an automated liquid handling system. In some cases, the support finds use in an automated liquid handling system for the high throughput isolation of cells, such as a flow cytometer.

In some embodiments of the method, the separating step includes applying an external magnetic field to immobilize a magnetic particle. Any convenient magnet may be used as a source of the external magnetic field (e.g., magnetic field gradient). In some cases, the external magnetic field is generated by a magnetic source, e.g. by a permanent magnet or electromagnet. In some cases, immobilizing the magnetic particles means the magnetic particles accumulate near the surface closest to the magnetic field gradient source, i.e. the magnet.

The separating may further include one or more optional washing steps to remove unbound material of the sample from the support. Any convenient washing methods may be used, e.g., washing the immobilized support with a biocompatible buffer which preserves the specific binding interaction of the polymeric dye and the specific binding member. Separation and optional washing of unbound material of the sample from the support provides for an enriched population of target cells where undesired cells and material may be removed.

In certain embodiments, the method further includes detecting the labeled target analyte. Detecting the labeled target analyte may include exciting the polymeric fluorescent tandem dye with one or more lasers and subsequently detecting fluorescence emission from the polymeric fluorescent tandem dye using one or more optical detectors. Detection of the labeled target can be performed using any convenient instruments and methods, including but not limited to, flow cytometry, FACS systems, fluorescence microscopy; fluorescence, luminescence, ultraviolet, and/or visible light detection using a plate reader; high performance liquid chromatography (HPLC); and mass spectrometry. When using fluorescently labeled components in the methods and compositions of the present disclosure, it is recognized that different types of fluorescence detection systems can be used to practice the subject methods. In some cases, high throughput screening can be performed, e.g., systems that use 96 well or greater microtiter plates. A variety of methods of performing assays on fluorescent materials can be utilized, such as those methods described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In some cases, excitation radiation, from an excitation source having a first wavelength, passes through excitation optics. The excitation optics cause the excitation radiation to excite the sample. In response, fluorescently labelled targets in the sample emit radiation which has a wavelength that is different from the excitation wavelength. Collection optics then collect the emission from the sample. The device can include a temperature controller to maintain the sample at a specific temperature while it is being scanned. In certain instances, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In some embodiments, the method of evaluating a sample for the presence of a target analyte further includes detecting fluorescence in a flow cytometer. In some embodiments, the method of evaluating a sample for the presence of a target analyte further includes imaging the labelling composition contacted sample using fluorescence microscopy. Fluorescence microscopy imaging can be used to identify a polymeric dye conjugate-target analyte binding complex in the contacted sample to evaluate whether the target analyte is present. Microscopy methods of interest that find use in the subject methods include laser scanning confocal microscopy.

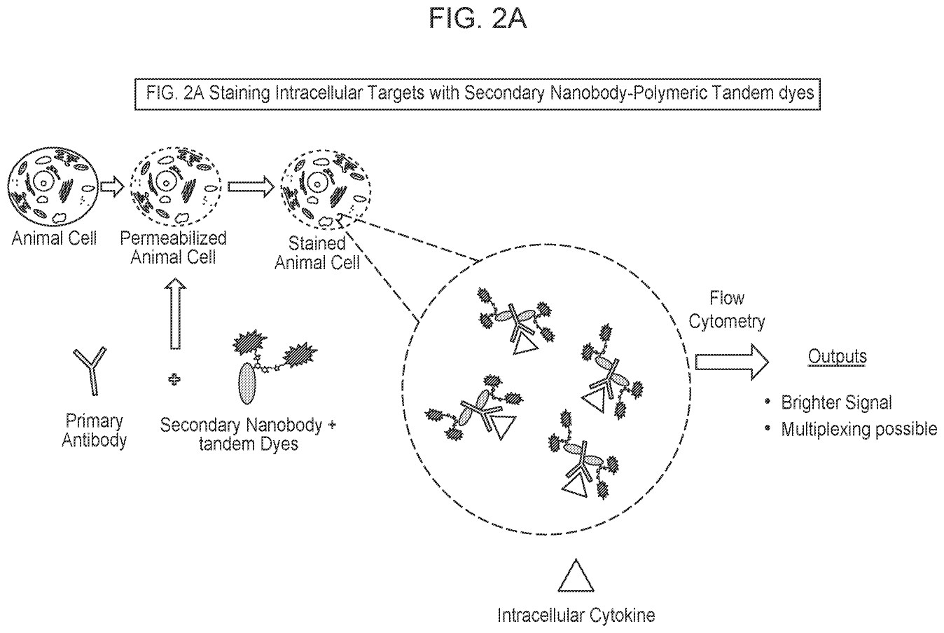

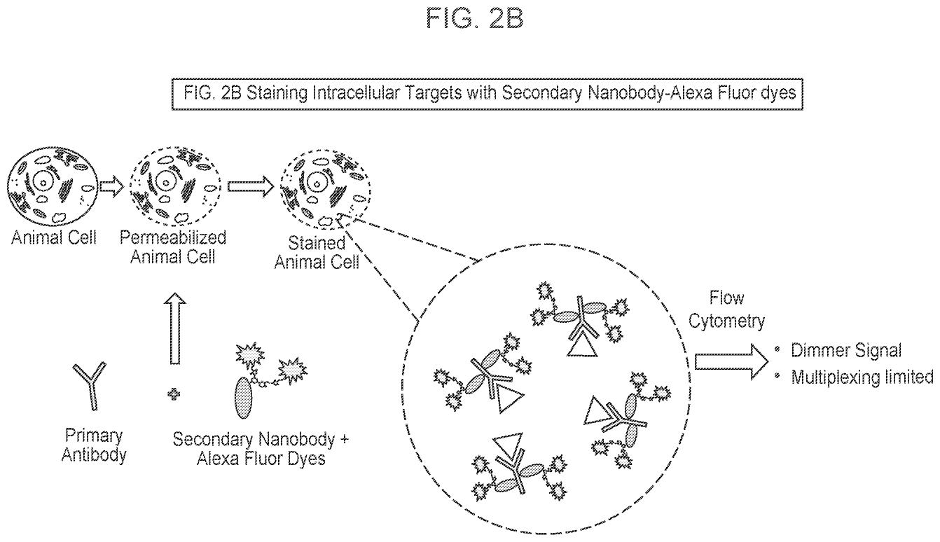

FIG. 2A provides an schematic illustration of an embodiment of the methods where a primary antibody is employed in conjunction with a single domain antibody/polymeric fluorescent tandem dye conjugate that specifically binds to the primary antibody to detect an intracellular cytokine. FIG. 2B illustrations an analogous method in which an Alexa-fluor labeled nanobody is employed.

Also provided are methods of producing a single domain antibody/polymeric fluorescent tandem dye conjugate, e.g., as described herein. In some embodiments, the method includes: contacting a single domain antibody with a polymeric fluorescent tandem dye (e.g., as described herein) to produce a single domain antibody/polymeric fluorescent tandem dye conjugate, wherein the polymeric fluorescent tandem dye includes a conjugation tag that covalently links the dye to the single domain antibody. The term “conjugation tag” refers to a group that includes a chemoselective functional group (e.g., as described herein) that can covalently link with a compatible functional group of a single domain antibody, after optional activation and/or deprotection. Any convenient conjugation tags may be utilized in the subject polymeric dyes in order to conjugate the polymeric fluorescent tandem dye to a single domain antibody of interest. In some embodiments, the conjugation tag includes a terminal functional group selected from an amino, a carboxylic acid or a derivative thereof, a thiol, a hydroxyl, a hydrazine, a hydrazide, a azide, an alkyne and a protein reactive group (e.g. amino-reactive, thiol-reactive, hydroxyl-reactive, imidazolyl-reactive or guanidinyl-reactive). Any convenient methods and reagents may be adapted for use in the subject methods in order to covalently link the conjugation tag to the single domain antibody. Methods of interest for labelling a target, include but are not limited to, those methods and reagents described by Hermanson, Bioconjugate Techniques, Third edition, Academic Press, 2013. The contacting step may be performed in an aqueous solution. In some instances, the conjugation tag includes an amino functional group and the target molecule includes an activated ester functional group, such as a NHS ester or sulfo-NHS ester, or vice versa. In certain instances, the conjugation tag includes a maleimide functional group and the target molecule includes a thiol functional group, or vice versa. In certain instances, the conjugation tag includes an alkyne (e.g., a cyclooctyne group) functional group and the target molecule includes an azide functional group, or vice versa, which can be conjugated via Click chemistry. In some cases, the method includes a separating step where the product single domain antibody/polymeric fluorescent tandem dye conjugate is separated from the reaction mixture, e.g., excess reagents or unlabeled single domain antibody. A variety of methods may be utilized to separate a target from a sample, e.g., via immobilization on a support, precipitation, chromatography, and the like.

In some instances, the method further includes detecting and/or analyzing the product single domain antibody/polymeric fluorescent tandem dye conjugate. In some instances, the method further includes fluorescently detecting the single domain antibody/polymeric fluorescent tandem dye conjugate. Any convenient methods may be utilized to detect and/or analyze the labelled target molecule in conjunction with the subject methods and compositions. Methods of analyzing a target of interest that find use in the subject methods, include but are not limited to, flow cytometry, fluorescence microscopy, in-situ hybridization, enzyme-linked immunosorbent assays (ELISAs), western blot analysis, magnetic cell separation assays and fluorochrome purification chromatography. Detection methods of interest include but are not limited to fluorescence spectroscopy, fluorescence microscopy, nucleic acid sequencing, fluorescence in-situ hybridization (FISH), protein mass spectroscopy, flow cytometry, and the like.

Detection may be achieved directly via the polymeric tandem dye, or indirectly by a secondary detection system. The latter may be based on any one or a combination of several different principles including, but not limited to, antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like). Suitable reporter molecules may be those known in the field of immunocytochemistry, molecular biology, light, fluorescence, and electron microscopy, cell immunophenotyping, cell sorting, flow cytometry, cell visualization, detection, enumeration, and/or signal output quantification. More than one antibody of specific and/or non-specific nature might be labelled and used simultaneously or sequentially to enhance target detection, identification, and/or analysis.

### Systems

Aspects of the invention further include systems for use in practicing the subject methods and compositions. A sample analysis system can include sample field of view or a flow channel loaded with a sample and single domain antibody/polymeric fluorescent tandem dye conjugate. In some embodiments, the system is a flow cytometric system including: a flow cytometer including a flow path; a composition in the flow path, wherein the composition includes: a sample; and a single domain antibody/polymeric fluorescent tandem dye conjugate (e.g., as described herein). In some embodiments, the system for analyzing a sample is a fluorescence microscopy system, including: a fluorescence microscope comprising a sample field of view; and a composition disposed in the sample field of view, wherein the composition comprises a sample; and a labelled specific binding member (e.g., as described herein).

In certain embodiments of the systems, the composition further includes a second specific binding member that is support bound and specifically binds the target analyte. In some cases, the support includes a magnetic particle. As such, in certain instances, the system may also include a controllable external paramagnetic field configured for application to an assay region of the flow channel.

The sample may include a cell. In some instances, the sample is a cell-containing biological sample. In some instances, the sample includes a labelled specific binding member specifically bound to a target cell. In certain instances, the target analyte that is specifically bound by the specific binding member is a cell surface marker of the cell. In certain cases, the cell surface marker is selected from a cell receptor and a cell surface antigen.

In certain aspects, the system may also include a light source configured to direct light to an assay region of the flow channel or sample field of view. The system may include a detector configured to receive a signal from an assay region of the flow channel or a sample field of view, wherein the signal is provided by the fluorescent composition. Optionally further, the sample analysis system may include one or more additional detectors and/or light sources for the detection of one or more additional signals.

In certain aspects, the system may further include computer-based systems configured to detect the presence of the fluorescent signal. A “computer-based system” refers to the hardware means, software means, and data storage means used to analyze the information of the present invention. The minimum hardware of the computer-based systems of the present invention includes a central processing unit (CPU), input means, output means, and data storage means. A skilled artisan can readily appreciate that any one of the currently available computer-based system are suitable for use in the subject systems. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In certain aspects, the system includes a flow cytometer. Suitable flow cytometry systems may include, but are not limited to, those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSCanto™ II flow cytometer, BD Accuri™ flow cytometer, BD Accuri™ 06 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like.

In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety.

In certain instances, flow cytometry systems of the invention are configured for imaging particles in a flow stream by fluorescence imaging using radiofrequency tagged emission (FIRE), such as those described in Diebold, et al. Nature Photonics Vol. 7(10); 806-810 (2013) as well as described in U.S. Pat. Nos. 9,423,353; 9,784,661; 9,983,132; 10,006,852; 10,078,045; 10,036,699; 10,222,316; 10,288,546; 10,324,019; 10,408,758; 10,451,538; 10,620,111; and U.S. Patent Publication Nos. 2017/0133857; 2017/0328826; 2017/0350803; 2018/0275042; 2019/0376895 and 2019/0376894 the disclosures of which are herein incorporated by reference.

Other systems may find use in practicing the subject methods. In certain aspects, the system may be a fluorimeter or microscope loaded with a sample having a fluorescent composition of any of the embodiments discussed herein. The fluorimeter or microscope may include a light source configured to direct light to the assay region of the flow channel or sample field of view. The fluorimeter or microscope may also include a detector configured to receive a signal from an assay region of the flow channel or field of view, wherein the signal is provided by the fluorescent composition.

### Kits

Aspects of the invention further include kits for use in practicing the subject methods and compositions. The compositions of the invention can be included as reagents in kits either as starting materials or provided for use in, for example, the methodologies described above. A kit can include single domain antibody/polymeric fluorescent tandem dye conjugate (e.g., as described herein) and a container. The single domain antibody/polymeric fluorescent tandem dye conjugate may be provided as a composition of multiple copies of the single domain antibody/polymeric fluorescent tandem dye conjugate. In such instances the polydispersity among the multiple copies of the single domain antibody/polymeric fluorescent tandem dye conjugate may be low. Any convenient containers can be utilized, such as tubes, bottles, or wells in a multi-well strip or plate, a box, a bag, an insulated container, and the like. The subject kits can further include one or more components selected from a primer specific binding ember for a given target analyte, a support bound specific binding member, a cell, a support, a biocompatible aqueous elution buffer, and instructions for use. A given kit may include reagents suitable for a detection of a single target analyte, or multiple reangest suitable for detection of two or more different target analytes, e.g., where a given kit is configured for multiplex detection applications.

In certain embodiments, the kit finds use in evaluating a sample for the presence of a target analyte, such as an intracellular target. As such, in some instances, the kit includes one or more components suitable for permeabilizing or lysing cells. The one or more additional components of the kit may be provided in separate containers (e.g., separate tubes, bottles, or wells in a multi-well strip or plate).

In certain aspects, the kit further includes reagents for performing a flow cytometric assay. Reagents of interest include, but are not limited to, buffers for reconstitution and dilution, buffers for contacting a cell sample the chromophore, wash buffers, control cells, control beads, fluorescent beads for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The compositions of the kit may be provided in a liquid composition, such as any suitable buffer. Alternatively, the compositions of the kit may be provided in a dry composition (e.g., may be lyophilized), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Utility

The single domain antibody/polymeric fluorescent tandem dye conjugates, compositions, methods and systems as described herein may find use in a variety of applications, including diagnostic and research applications, in which the labelling, detection and/or analysis of a target of interest is desirable. Such applications include methodologies such as cytometry, microscopy, immunoassays (e.g. competitive or non-competitive), assessment of a free analyte, assessment of receptor bound ligand, and so forth. The compositions, system and methods described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the compositions and methods find use in applications where the evaluation of a sample for the presence of a target analyte is of interest.

In some cases, the methods and compositions find use in any assay format where the detection and/or analysis of a target from a sample is of interest, including but not limited to, flow cytometry, fluorescence microscopy, in-situ hybridization, enzyme-linked immunosorbent assays (ELISAs), western blot analysis, magnetic cell separation assays and fluorochrome purification chromatography. In certain instances, the methods and compositions find use in any application where the fluorescent labelling of a target molecule is of interest.

The subject compositions may be adapted for use in any convenient applications where pairs of specific binding members find use, such as biotin-streptavidin and hapten-anti-hapten antibody.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### I. Reagent Preparation

### A. VHH Anti-Mouse IgG1-RB780 Preparation:

Tandem Dye conjugation to VHH anti-mouse IgG1 (Chromotek Nanobodies CTK103, CTK 104)

RB780™ tandem dye was modified to contain a terminal maleimide group for maleimide-thiol attachment to N-terminal and C-terminal cysteines of a target engineered nanobody CTK103 (Chromotek). The resulting purified RB780-maleimide dye was then added to a buffer solution (10 mM HEPES pH 7.0 500 mM NaCl) at a concentration of 20 mg/mL. Nanobody was received frozen in aqueous buffer (10 mM HEPES pH 7.0, 500 mM NaCl, 1 mM TCEP). Prior to the conjugation, the frozen nanobody solution was thawed to 4° C., centrifuged and the supernatant liquid was transferred to a reaction vessel. A volume of RB780-maleimide dye solution corresponding to 2.7 molar equivalents of nanobody was then added to the solution containing the thawed nanobody. The mixture was vortexed, placed in an ice bath and agitated with magnetic stirring for 2 hours. The resulting crude reaction mixture was then purified using a Superdex SEC column with a standard elution buffer. Fractions of desired product were pooled and characterized. Equimolar amounts of CTK103-RB780 conjugate and CTK104-RB780 were combined in solution prior to staining of the primary antibodies.

RB780™ was modified to contain a terminal maleimide group for maleimide-thiol attachment to N-terminal and C-terminal cysteines of a target engineered nanobody CTK104 (Chromotek). The resulting purified RB780-maleimide dye was then added to a buffer solution (10 mM HEPES pH 7.0 500 mM NaCl) at a concentration of 20 mg/mL. Nanobody was received frozen in aqueous buffer (10 mM HEPES pH 7.0, 500 mM NaCl, 1 mM TCEP). Prior to the conjugation, the frozen nanobody solution was thawed to 4° C., centrifuged and the supernatant liquid was transferred to a reaction vessel. A volume of RB780-maleimide dye solution corresponding to 2.7 molar equivalents of nanobody was then added to the solution containing the thawed nanobody. The mixture was vortexed, placed in an ice bath and agitated with magnetic stirring for 2 hours. The resulting crude reaction mixture was then purified using a Superdex SEC column with a standard elution buffer. Fractions of desired product were pooled and characterized. Equimolar amounts of CTK103-RB780 conjugate and CTK104-RB780 were combined in solution prior to staining of the primary antibodies.

### B. VHH Anti-Mouse IgG2b-RB780 Preparation:

Tandem Dye conjugation to VHH anti-mouse IgG2b (Chromotek Nanobodies CTK105, CTK 106)

RB780™ was modified to contain a terminal maleimide group for maleimide-thiol attachment to N-terminal and C-terminal cysteines of a target engineered nanobody CTK105 (Chromotek). The resulting purified RB780-maleimide dye was then added to a buffer solution (10 mM HEPES pH 7.0 500 mM NaCl) at a concentration of 20 mg/mL. Nanobody was received frozen in aqueous buffer (10 mM HEPES pH 7.0, 500 mM NaCl, 1 mM TCEP). Prior to the conjugation, the frozen nanobody solution was thawed to 4° C., centrifuged and the supernatant liquid was transferred to a reaction vessel. A volume of RB780-maleimide dye solution corresponding to 2.7 molar equivalents of nanobody was then added to the solution containing the thawed nanobody. The mixture was vortexed, placed in an ice bath and agitated with magnetic stirring for 2 hours. The resulting crude reaction mixture was then purified using a Superdex SEC column with a standard elution buffer. Fractions of desired product were pooled and characterized. Equimolar amounts of CTK105-RB780 conjugate and CTK106-RB780 were combined in solution prior to staining of the primary antibodies.

RB780™ was modified to contain a terminal maleimide group for maleimide-thiol attachment to N-terminal and C-terminal cysteines of a target engineered nanobody CTK106 (Chromotek). The resulting purified RB780-maleimide dye was then added to a buffer solution (10 mM HEPES pH 7.0 500 mM NaCl) at a concentration of 20 mg/mL. Nanobody was received frozen in aqueous buffer (10 mM HEPES pH 7.0, 500 mM NaCl, 1 mM TCEP). Prior to the conjugation, the frozen nanobody solution was thawed to 4° C., centrifuged and the supernatant liquid was transferred to a reaction vessel. A volume of RB780-maleimide dye solution corresponding to 2.7 molar equivalents of nanobody was then added to the solution containing the thawed nanobody. The mixture was vortexed, placed in an ice bath and agitated with magnetic stirring for 2 hours. The resulting crude reaction mixture was then purified using a Superdex SEC column with a standard elution buffer. Fractions of desired product were pooled and characterized. Equimolar amounts of CTK105-RB780 conjugate and CTK106-RB780 were combined in solution prior to staining of the primary antibodies.

### II. Further Studies

### A. Preparing of RB780 Labeled Nanobodies

### 1. Modifying RB780

22 milligrams of a RB780 derivative bearing an amine reactive site was dissolved in degassed, 1× phosphate buffered saline (VWR catalog #9706-660) at a concentration of 10 mg/mL. 5.55 milligrams of SMCC (succinimidyl 4-(N-maleimidomethyl)cyclohexane-1-carboxylate) was dissolved in degassed, amine free DMF (Fisher Scientific catalog #AA43465K7). The SMCC solution was added to the solution containing RB780-amine and stirred at room temperature for 2 hours. The crude reaction mixture was purified by size exclusion membrane (MilliporeSigma™ UFC900308) to remove unreacted SMCC, and freeze-dried on a Schlenk line overnight. Yield and purity were assessed by ion exchange chromatography (Thermo Scientific Ultimate 3000 with Propoac SAX-10 4×250 mm).

### 2. Reduction of Nanobody Disulfides

Nanobody samples previously stored in buffer at −80 C were thawed and buffer exchanged into 10 mM Hepes pH 7.0, 500 mM NaCl, 1 mM TCEP using size exclusion membrane (MilliporeSigma™ UFC900308). The buffer exchanged solution containing freshly reduced nanobodies was then centrifuged to remove precipitates and aggregates.

### 3. Dye Conjugation to Nanobodies

Nanobody CTK0103 (ProteinTech) was concentrated to 2 mg/mL in 10 mM Hepes pH 7.0, 500 mM NaCl, 1 mM TCEP and kept below 4 C via ice bath. RB780-maleimide was dissolved at 20 mg/mL in amine free DMF (Fisher Scientific catalog #AA43465K7) and cooled to ˜4 C via ice bath. The RB780-maleimide solution was added to the nanobody solution at a ratio of 2.7 moles of RB780-maleimide per mole of nanobody and stirred at ˜4 C for two hours. The crude conjugation mixture was purified by size exclusion chromatography (GE AKTA FPLC with Superdex) and characterized by SDS-PAGE & UV-Vis.

Nanobody CTK0104 (ProteinTech) was concentrated to 2 mg/mL in 10 mM Hepes pH 7.0, 500 mM NaCl, 1 mM TCEP and kept below 4 C via ice bath. RB780-maleimide was dissolved at 20 mg/mL in amine free DMF (Fisher Scientific catalog #AA43465K7) and cooled to −4 C via ice bath. The RB780-maleimide solution was added to the nanobody solution at a ratio of 2.7 moles of RB780-maleimide per mole of nanobody and stirred at ˜4 C for two hours. The crude conjugation mixture was purified by size exclusion chromatography (GE AKTA FPLC with Superdex) and characterized by SDS-PAGE & UV-Vis.

Nanobody CTK0105 (ProteinTech) was concentrated to 2 mg/mL in 10 mM Hepes pH 7.0, 500 mM NaCl, 1 mM TCEP and kept below 4 C via ice bath. RB780-maleimide was dissolved at 20 mg/mL in amine free DMF (Fisher Scientific catalog #AA43465K7) and cooled to ˜4 C via ice bath. The RB780-maleimide solution was added to the nanobody solution at a ratio of 2.7 moles of RB780-maleimide per mole of nanobody and stirred at ˜4 C for two hours. The crude conjugation mixture was purified by size exclusion chromatography (GE AKTA FPLC with Superdex) and characterized by SDS-PAGE & UV-Vis.

Nanobody CTK0106 (ProteinTech) was concentrated to 2 mg/mL in 10 mM Hepes pH 7.0, 500 mM NaCl, 1 mM TCEP and kept below 4 C via ice bath. RB780-maleimide was dissolved at 20 mg/mL in amine free DMF (Fisher Scientific catalog #AA43465K7) and cooled to ˜4 C via ice bath. The RB780-maleimide solution was added to the nanobody solution at a ratio of 2.7 moles of RB780-maleimide per mole of nanobody and stirred at ˜4 C for two hours. The crude conjugation mixture was purified by size exclusion chromatography (GE AKTA FPLC with Superdex) and characterized by SDS-PAGE & UV-Vis.

### B. Cell Prep/Staining & Flow Cytometry

### 1. Surface Staining

100 μl of well-mixed anticoagulated whole blood was incubated with the unlabeled primary antibody, human CD4 (clone SK3, a mouse IgG1), in the dark at room temperature for 20-30 minutes. 2 ml of lysing solution was then added before incubating in the dark at room temperature. After 10 minutes, the cells were washed and the secondary reagent, nanobody anti-mouse IgG1-RB780 was added (nanobodies CTK0103 and CTK0104 from ProteinTech), before incubating in the dark at room temperature for 20-30 minutes. A final wash was performed and 200-500 ul of wash buffer were added, prior to flow cytometric analysis, or before storage in the refrigerator at 2-8° C.

100 μl of well-mixed anticoagulated whole blood was incubated with the unlabeled primary antibody, human CD56 (clone NCAM16.2, a mouse IgG2b), in the dark at room temperature for 20-30 minutes. 2 ml of lysing solution was then added before incubating in the dark at room temperature. After 10 minutes, the cells were washed and the secondary reagent, nanobody VHH anti-mouse IgG2b-RB780 (nanobodies CTK0105 and CTK0106 from ProteinTech) was added, before incubating in the dark at room temperature for 20-30 minutes. A final wash was performed and 200-500 ul of wash buffer were added, prior to flow cytometric analysis, or before storage in the refrigerator at 2-8° C.

### 2. Intracellular Staining

Peripheral Blood Mononuclear Cells (PBMCs) was activated, before fixing and permeabilizing according to standard protocols. 100 μl of PBMCs was incubated with the unlabeled primary antibody, human IFN-γ (clone B27, a mouse IgG1), at 4° C. for 30 minutes in the dark. The cells were then washed twice before adding the secondary reagent, nanobody VHH anti-mouse IgG1-RB780 (nanobodies CTK0103 and CTK0104 from ProteinTech) followed by incubating at 4° C. for 30 minutes in the dark. A final wash was performed and 200-500 ul of wash buffer were added, prior to flow cytometric analysis, or before storage in the refrigerator at 2-8° C.

### C. Results:

### 1. Surface Staining

We observed a positive staining in the lymphocytes corresponding to the CD4 positive population. The signal was dimmer when using the nanobodies CTK0103 and CTK0104 (either individually used or pooled together), compared to the signal observed with existing same nanobodies conjugated with AlexaFluor647 (CTK0103 and CTK0104 pooled from ProteinTech). The comparison was run both in mass equivalent and in mole equivalent.

We also observed a positive staining in the lymphocytes corresponding to the CD56 positive population. The signal was dimmer when using the nanobodies CTK0105 and CTK0106 (either individually used or pooled together), compared to the signal observed with existing same nanobodies conjugated with AlexaFluor647 (CTK0105 and CTK0106 pooled from ProteinTech). The comparison was run both in mass equivalent and in mole equivalent.

### 2. Intracellular Staining:

We observed a positive staining in the lymphocytes corresponding to the IFN-γ positive population. The signal was dimmer when using the nanobodies CTK0103 and CTK0104 (either individually used or pooled together), compared to the signal observed with existing same nanobodies conjugated with AlexaFluor647 (CTK0103 and CTK0104 pooled from ProteinTech). The comparison was run both in mass equivalent and in mole equivalent.

### III. Additional Observations

Single domain antibody/polymeric fluorescent tandem dye conjugates of embodiments of the invention provide for a number of advantages. Prior to the work reported herein, there have been two general classes of commercialized sdAb+fluorescent dye reagents. The first consists of sdAb+a fluorescent protein such as GFP, GYP, GRP etc. Typically these reagents are produced recombinantly as fusion proteins. While these reagents do retain advantages in manufacturability and performance because of their small size and in vivo expression, their utility is limited because they are relatively dim, and because they are not tandem dyes and do not enable the multiplexing advantage of tandem dyes. The second class of sdAb+fluorescent dye reagents consists of a sdAb conjugated to a small molecule dye or a few small molecule dyes, such as AlexaFluor647. These reagents also have size based advantages but are limited in several ways. As reagents they have a brightness limit related to self quenching behavior. If too many AlexFluor647 dyes are attached to the sdAb, the fluorescence of the reagent can actually go down. Additionally, and like the fusion proteins described above, these small molecule dyes are not tandem dyes and do not enable the multiplexing advantage of tandem dyes.

Single domain antibody/polymeric fluorescent dye conjugate reagents of embodiments of the invention combine the benefits of small size and improved manufacture, with additional benefits which may include one or more of increased reagent brightness and compatibility with high parameter panel design. These advantages are achieved by using polymeric fluorescent tandem dyes instead of single molecule dyes. Polymeric fluorescent tandem dyes have higher per molecule brightness than small molecule dyes and do not exhibit the appreciable inter-dye quenching of small molecule dyes when conjugated to antibodies. Also, polymeric fluorescent tandem dyes represent a platform of tandem dyes which are intended specifically for optimized multicolor flow cytometry. As such, conjugates of the invention may be employed in multiplex applications using panels of dyes.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A single domain antibody/polymeric fluorescent tandem dye conjugate, the conjugate comprising:
(a) a single domain antibody; and
(b) one or more polymeric tandem fluorescent dyes conjugated to the single domain antibody, wherein the one or more polymeric tandem fluorescent dyes comprises:
(i) a polymeric backbone comprising non-conjugated repeat units;
(ii) a plurality of pendant donor fluorophores each independently linked to a non-conjugated repeat unit of the polymeric backbone; and
(iii) one or more pendant acceptor fluorophores linked to a non-conjugated repeat unit of the polymeric backbone, wherein pendant donor and acceptor fluorophores are in energy transfer relationship.

2. The conjugate according to claim 1, wherein the single domain antibody comprises a heavy chain or a light chain.

3. The conjugate according to claim 2, wherein the single domain antibody comprises a V_{H}H domain.

4. The conjugate according to claim 2, wherein the single domain antibody comprises a V_{NAR} domain.

5. The conjugate according to claim 1, wherein the single domain antibody specifically binds to an intracellular antigen.

6. The conjugate according to claim 1, wherein the single domain antibody specifically binds to cell surface antigen.

7. The conjugate according to claim 1, wherein the single domain antibody specifically binds to primary antibody.

8. The conjugate according to claim 1, wherein the conjugate comprises two or more polymeric tandem fluorescent dyes conjugated to the single domain antibody.

9. The conjugate according to claim 1, wherein the polymeric tandem fluorescent dye is described by the formula: 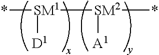
wherein:
the polymeric backbone of non-conjugated repeat units comprises SM¹ and SM² co-monomers that are each independently a non-conjugated co-monomer;
each D¹ is independently a pendant donor fluorophore linked to SM¹;
each A¹ is independently a pendant acceptor fluorophore linked to SM²;
x is 75 mol % or more; and
y is 25 mol % or less.

10. The conjugate according to claim 9, wherein the repeat units of the polymeric backbone have a defined linear sequence.

11. The conjugate according to claim 10, wherein SM¹ and SM² are co-monomers derived from amino acids and peptoid monomers.

12. (canceled)

13. The conjugate according to claim 1, wherein the polymeric backbone is a linear polymer.

14. The conjugate according to claim 1, wherein the pendant donor fluorophores are configured in energy-transferring proximity to each other.

15. The conjugate according to claim 1, wherein the polymeric tandem dye has a Stokes shift of 100 nm or more.

16. The conjugate according to claim 1, wherein the pendant donor fluorophores are selected from fused tricyclic aryl, fused tricyclic heteroaryl and BODIPY fluorophores.

17. The conjugate according to claim 16, wherein the pendant donor fluorophores are BODIPY fluorophores.

18. The conjugate according to claim 1, wherein the pendant donor fluorophores are substituted with a water-soluble group.

19. The conjugate according to claim 18, wherein the water soluble group comprises a polyethylene glycol.

20. The conjugate according to claim 1, wherein the acceptor fluorophore is a small molecule fluorophore.

21. The conjugate according to claim 20, wherein the acceptor fluorophore is selected from a cyanine dye, a rhodamine dye, a xanthene dye, a coumarin dye, a polymethine, a pyrene, a dipyrromethene borondifluoride, a napthalimide, a thiazine dye and an acridine dye.

22. A method of evaluating a sample for the presence of a target analyte, the method comprising:
(a) combining the sample with a single domain antibody/polymeric fluorescent tandem dye conjugate that specifically binds the target analyte to produce an assay composition, wherein the conjugate comprises:
(1) an single domain antibody; and
(2) one or more polymeric tandem fluorescent dyes conjugated to the single domain antibody, wherein the one or more polymeric tandem fluorescent dyes comprises:
(i) a polymeric backbone comprising non-conjugated repeat units;
(ii) a plurality of pendant donor fluorophores each independently linked to a non-conjugated repeat unit of the polymeric backbone; and
(iii) one or more pendant acceptor fluorophores linked to a non-conjugated repeat unit of the polymeric backbone, wherein pendant donor and acceptor fluorophores are in energy transfer relationship; and
(b) assaying the assay composition for the presence of any conjugate-target analyte binding complexes to evaluate whether the target analyte is present in the sample.

23. The method according to claim 22, wherein the target analyte is associated with a cell.

24. The method according to claim 23, wherein the target analyte is a cell surface marker.

25. The method according to claim 24, wherein the cell surface marker is selected from the group consisting of a cell receptor and a cell surface antigen.

26. The method according to claim 23, wherein the target analyte is an intracellular target.

27. The method according to claim 26, wherein the method further comprises permeabilizing the cell.

28. The method according to any of claims 22 to 27, wherein the assaying comprises flow cytometrically analyzing the assay composition.

29. The method according to any of claims 22 to 28, wherein the single domain antibody comprises a heavy chain domain or a light chain domain.

30. The method according to claim 29, wherein the single domain antibody comprises a V_{H}H domain.

31. The method according to claim 29, wherein the single domain antibody comprises a V_{NAR} domain.

32. The method according to any of claims 22 to 32, wherein the conjugate comprises two or more polymeric tandem fluorescent dyes conjugated to the single domain antibody.

33. The method according to any of claims 22 to 32, wherein the polymeric tandem fluorescent dye is described by the formula: 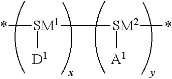
wherein:
the polymeric backbone of non-conjugated repeat units comprises SM¹ and SM² co-monomers that are each independently a non-conjugated comonomer;
each D¹ is independently a pendant donor fluorophore linked to SM¹;
each A¹ is independently a pendant acceptor fluorophore linked to SM²;
x is 75 mol % or more; and
y is 25 mol % or less.

34. The method according to claim 33, wherein the repeat units of the polymeric backbone have a defined linear sequence.

35. The method according to Claim 34, wherein SM¹ and SM² are comonomers derived from amino acids and peptoid monomers.

36. The method according to Claim 35, wherein the polymeric backbone is a polypeptide having a defined sequence of α-amino acid residues and/or β-amino acid residues.

37. The method according to any of claims 22 to 36, wherein the polymeric backbone is a linear polymer.

38. The method according to any of claims 22 to 37, wherein the pendant donor fluorophores are configured in energy-transferring proximity to each other.

39. The method according to any of claims 22 to 38, wherein the polymeric tandem fluorescent dye has a Stokes shift of 100 nm or more.

40. The method according to any of claims 22 to 39, wherein the pendant donor fluorophores are selected from fused tricyclic aryl, fused tricyclic heteroaryl and BODIPY fluorophores.

41. The method according to claim 40, wherein the pendant donor fluorophores are BODIPY groups.

42. The method according to any of claims 22 to 41, wherein the pendant donor fluorophores are substituted with a water-soluble group.

43. The method according to claim 42, wherein the water soluble group comprises a polyethylene glycol.

44. The method according to any of claims 22 to 43, wherein the acceptor fluorophore is a small molecule fluorophore.

45. The method according to claim 44, wherein the acceptor fluorophore is selected from a cyanine dye, a rhodamine dye, a xanthene dye, a coumarin dye, a polymethine, a pyrene, a dipyrromethene borondifluoride, a napthalimide, a thiazine dye and an acridine dye.

46. A kit comprising:
a single domain antibody/polymeric fluorescent tandem dye conjugate, the conjugate comprising:
(a) an single domain antibody; and
(b) one or more polymeric tandem fluorescent dyes conjugated to the single domain antibody, wherein the one or more polymeric tandem fluorescent dyes comprises:
(i) a polymeric backbone comprising non-conjugated repeat units;
(ii) a plurality of pendant donor fluorophores each independently linked to a non-conjugated repeat unit of the polymeric backbone; and
(iii) one or more pendant acceptor fluorophores linked to a non-conjugated repeat unit of the polymeric backbone, wherein pendant donor and acceptor fluorophores are in energy transfer relationship.

47. The kit according to claim 46, wherein single domain antibody comprises a heavy chain or a light chain.

48. The kit according to claim 47, wherein the single domain antibody comprises a VHH domain.

49. The kit according to claim 47, wherein the single domain antibody comprises a V_{NAR} domain.

50. The kit according to any of claims 46 to 49, wherein the single domain antibody specifically binds to an intracellular antigen.

51. The kit according to any of claims 46 to 50, wherein the single domain antibody specifically binds to cell surface antigen.

52. The kit according to any of claims 46 to 51, wherein the single domain antibody specifically binds to primary antibody.

53. The kit according to any of claims 46 to 52, wherein the conjugate comprises two or more polymeric tandem fluorescent dyes conjugated to the single domain antibody.

54. The kit according to any of claims 46 to 53, wherein the polymeric tandem fluorescent dye is described by the formula: 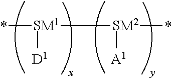
wherein:
the polymeric backbone of non-conjugated repeat units comprises SM¹ and SM² co-monomers that are each independently a non-conjugated comonomer;
each D¹ is independently a pendant donor fluorophore linked to SM¹;
each A¹ is independently a pendant acceptor fluorophore linked to SM²;
x is 75 mol % or more; and
y is 25 mol % or less.

55. The kit according to claim 54, wherein the repeat units of the polymeric backbone have a defined linear sequence.

56. The kit according to claim 55, wherein SM¹ and SM² are co-monomers derived from amino acids and peptoid monomers.

57. The kit according to claim 56, wherein the polymeric backbone is a polypeptide having a defined sequence of α-amino acid residues and/or β-amino acid residues.

58. The kit according to any of claims 46 to 57, wherein the polymeric backbone is a linear polymer.

59. The kit according to any of claims 46 to 58, wherein the pendant donor fluorophores are configured in energy-transferring proximity to each other.

60. The kit according to any of claims 46 to 59, wherein the polymeric tandem fluorescent dye has a Stokes shift of 100 nm or more.

61. The kit according to any of claims 46 to 60, wherein the pendant donor fluorophores are selected from fused tricyclic aryl, fused tricyclic heteroaryl and BODIPY fluorophores.

62. The kit according to claim 61, wherein the pendant donor fluorophores are BODIPY groups.

63. The kit according to any of claims 46 to 62, wherein the pendant donor fluorophores are substituted with a water-soluble group.

64. The kit according to claim 63, wherein the water soluble group comprises a polyethylene glycol.

65. The kit according to any of claims 46 to 64, wherein the acceptor fluorophore is a small molecule fluorophore.

66. The kit according to claim 65, wherein the acceptor fluorophore is selected from a cyanine dye, a rhodamine dye, a xanthene dye, a coumarin dye, a polymethine, a pyrene, a dipyrromethene borondifluoride, a napthalimide, a thiazine dye and an acridine dye.
